(19) United States
(12) Patent Application Publication
(10) Pub. No.: US 2010/0210602 A1
(43) Pub. Date: Aug. 19, 2010

(54) PhoU (PerF), A PERSISTENCE SWITCH INVOLVED IN PERSISTER FORMATION AND TOLERANCE TO MULTIPLE ANTIBIOTICS AND STRESSES AS A DRUG TARGET FOR PERSISTER BACTERIA

(75) Inventors: Ying Zhang, Baltimore, MD (US); Yongfang Li, Baltimore, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/519,021

(22) PCT Filed: Dec. 12, 2007
(86) PCT No.: PCT/US07/25365

§ 371 Date: Apr. 26, 2010

Related U.S. Application Data
(60) Provisional application No. 60/874,399, filed on Dec. 12, 2006.

(51) Int. Cl. A61K 31/65; C12N 1/20; A61K 31/495; C12Q 1/42; A61K 31/506; A61K 31/192; A61P 31/04

(52) U.S. Cl. 514/152; 435/252.1; 514/252.12; 435/21; 514/256; 514/568

(57) ABSTRACT

The PhoU protein is a widely expressed protein in bacteria, but not in eukaryotes. The PhoU protein is required for persister formation in bacteria. The invention includes compositions to reduce persister formation and their use as therapeutic agents. The invention further includes methods for identification of compounds to reduce persister formation. The invention further includes kits for the identification of agents that modulate the activity and expression of PhoU.

### REFERENCE TO RELATED APPLICATIONS

This application claims priority to U.S. Provisional Patent Application Ser. No. 60/874,399 filed on Dec. 12, 2006, which is incorporated herein in its entirety.

### STATEMENT AS TO FEDERALLY SUPPORTED RESEARCH

The present invention was made with United States government support under National Institutes of Health (NIH) grant numbers AI044063 and AI049485. Accordingly, the United States government has certain rights to the invention.

### BACKGROUND

The phenomenon of bacterial persistence was first described by Joseph Bigger in 1944 when he found that penicillin could not completely sterilize Staphylococcal cultures in vitro (J. W. Bigger, Lancet II, 497 (1944)). The residual small number of persister bacteria not killed by the antibiotic were still susceptible to the same antibiotic upon subculture in fresh medium. The nonsusceptibility or tolerance to antibiotics (and stresses) in persisters is physiological or phenotypic and distinct from stable genetic resistance (K. Lewis, Biochemistry (Mose). 70, 267 (2005); Y. Zhang, Ann. Rev. Pharmacol. Toxicol, 45, 529 (2005)). The persister bacteria have been found to be due to pre-existing slow growing metabolically quiescent bacteria that are not susceptible to antibiotics (N. Q. Balaban et al., Science 305, 1622 (2004)). In log phase cultures there is only a very small number of persister bacteria presumably due to carry-over from the inoculum, but the number of persisters increases as the cultures enter stationary phase. The persister phenomenon is presumably a protective strategy bacteria deploy to survive as a species under adverse conditions such as starvation, stress and antibiotic exposure. The persister bacteria present in biofilms (P. S. Stewart, Mt. J. Med. Microbiol. 292, 107 (2002) and also during natural infection process in the host with or without antibiotic treatment (W. McDermott, Yale J. Biol. Med. 30, 257 (1958)) pose a formidable challenge for effective control of a diverse range of bacterial infections such as that caused by Mycobacterium tuberculosis.

Despite the original discovery of the persister phenomenon over 60 years ago, the mechanism behind bacterial persistence has been elusive as the persisters represent a small fraction of the bacterial population and constantly changing. The rare bacterial population phenomenon and its fluctuating nature have made the problem of bacterial persistence almost intractable and pose significant intellectual and practical challenges. The first molecular study of bacterial persistence was carried out by Moyed and colleagues in 1983 when a gene in E. coli called hipA was identified whose mutation caused about 100-1000 fold increase in penicillin tolerant persister bacteria (H. S. Moyed, K. P. Bertrand, J. Bacteriol. 155, 768 (1983)). hipA forms an operon with hipB and is thought to be a toxin-antitoxin (TA) module where HipA as a toxin is tightly regulated by repressor HipB, which forms a complex with HipA (D. S. Black, B. Irwin, H. S. Moyed, J. Bacteriol. 176, 4081 (1994)). The hipA7 mutant contains two mutations (G22S and D291A) (S. B. Korch, T. A. Henderson, T. M. Hill, Mol. Microbiol. 50, 1199 (2003)) involved in persistence to different antibiotics and also some stress conditions (R. Scherrer, H. S. Moyed, I Bacteriol. 170, 3321 (1988); T. J. Falla, I. Chopra, Antimicrob. Agents Chemother. 42, 3282 (1998)). Exactly how hipA7 mediates persister formation is unclear. Most recently, HipA has been shown to be a serine kinase and in contrast to wild type HipA, mutant HipA did not confer tolerance to antibiotics when overexpressed (F. F. Correia at al., J. Bacteriol. October 13; [Epub ahead of print] (2006)). Given the significance of HipAB in bacterial persistence in some Gram-negative bacteria that have HipA homolog, it cannot explain the universal persister phenomenon in some other Gram-negative bacteria and especially Gram-positive bacteria that do not have HipA homologs.

Based on the microarray analysis of E. coli persisters not killed by ampicillin, Lewis and colleagues proposed a model for persisters where persister formation is dependent on various TA modules such as HipBA and ReIBE, which can inhibit peptidoglycan, RNA and DNA synthesis, and protein synthesis, respectively (K. Pedersen et al. Cell 112, 131 (2003)), leading to multidrug tolerance (MDT) (I. Keren et al., J. Bacteriol. 186, 8172 (2004). Overexpression of several toxins such as HipA, ReIE, and MazF (N. Vazquez-Laslop, H. Lee, A. A. Neyfakh, 1 Bacteriol. 188, 3494 (2006); S. B. Korch, T. M. Hill,” Bacteriol. 188, 3826 (2006)) were found to increase persister formation. However, a recent study showed that overexpression of unrelated toxic proteins such as heat shock protein DnaJ and PrmC. also caused higher persister formation. This finding challenges the significance of the TA modules as a specific and universal mechanism for persister formation.

### SUMMARY OF THE INVENTION

The invention includes methods to decrease persister formation and/or increase killing of a bacterial cell by administration of an agent that inhibits the activity of a PhoU protein or a PhoU homolog in a PhoU-containing bacteria. In an embodiment, the agent is an inhibitor of a PhoU phosphatase activity in a PhoU containing bacteria. In an embodiment, the inhibitor of PhoU phosphatase activity is one or more of piperazine, pyrantel pamoate, and tetracyclines including meclocycline and doxycycline, or aurintriccarboxylic acid. In an embodiment, the agent decreases PhoU phosphatase activity by decreasing expression of a PhoU phosphatase in a bacterial cell by decreasing transcription or translation of a phoU gene or a PhoU protein, respectively.

The invention includes methods to decrease persister formation and/or increase killing of a bacterial cell by administration of an agent that increases metabolic activity and/or increases phosphate concentration in a bacteria. Increased phosphate concentration in bacterial cells is known to decrease PhoU phosphatase activity.

The invention includes methods for identification of an agent to decrease persister formation and/or increase killing of a bacterial cell by contacting a PhoU phosphatase with an agent and detecting a change in the phosphatase level of a PhoU phosphatase as compared to a control not contacted with an agent. In an embodiment, agents from a compound library are screened. In an embodiment, the PhoU phosphatase is in a cell free system, isolated and removed from a cell. In an embodiment, the PhoU phosphatase activity is expressed in a cell, either heterologously or in a cell that expresses the protein endogenously. In an embodiment, PhoU phosphatase activity is detected using an in vitro phosphatase activity assay. In an embodiment, PhoU phosphatase activity is detected by increased cell killing as compared to a control cell not contacted with the agent. In an embodiment, cell killing is assayed in a stationary phase culture. In an embodiment, cell killing is assayed in a log phase culture.

The invention further includes a method for prevention, amelioration, or treatment of a bacterial infection to decrease persister formation and/or increase killing of a bacterial cell by administration of an agent that decreases phosphatase activity of a PhoU phosphatase. In an embodiment, the agent is an inhibitor of a PhoU phosphatase activity in a PhoU containing bacteria. In an embodiment, the inhibitor of PhoU phosphatase activity is one or more of piperazine, pyrantel pamoate, a tetracycline such as meclocycline or doxycycline, or aurintricarboxylic acid. In an embodiment, the agent decreases PhoU phosphatase activity by decreasing expression of a PhoU phosphatase in a bacterial cell.

The invention includes the use of a PhoU phosphatase activity inhibitor as an adjuvant in combination with an antibacterial agent for the treatment of a bacterial infection. In an embodiment, the adjuvant therapy results in a decrease in relapse. PhoU is a ubiquitous enzyme present in virtually all bacterial species and can serve as a target for intervention to reduce or eliminate persister bacteria for improved prevention, amelioration, and treatment of bacterial infection as an adjuvant with one or more antibiotics. In an embodiment, the infection is a bacterial infection by E. coli, Mycobacterium tuberculosis, Pseudomonas aeruginosa, or any Staphlococcal or Streptococcal bacteria. In an embodiment, the adjuvant is one or more of piperazine, pyrantel pamoate, a tetracycline such as meclocycline or doxycycline, or aurintricarboxylic acid. In an embodiment, the antibacterial agent is one or more of beta-lactams or cephalosporins, daptomycin, aminoglycosides, macrolides-lincosamides-streptogramins, linezolid, tetracylcines and quinolones, sulfa drugs, isoniazid (INH), rifampicin (RIF), or pyrazinamide (PZA), or any combination thereof.

The invention further includes a pharmaceutically acceptable antibacterial agent in combination with a pharmaceutically acceptable PhoU phosphatase activity inhibitor. In an embodiment, the antibiotic is selected from the group consisting of beta-lactams or cephalosporins, daptomycin, aminoglycosides, macrolides-lincosamides-streptogramins, linezolid, tetracylcines and quinolones, sulfa drugs, INH, RIF, and PZA, or any combination thereof; and the pharmaceutically acceptable PhoU phosphatase inhibitor is selected from the group consisting of, piperazine, pyrantel pamoate, tetracyclines such as meclocycline, and doxycycline; or any combination thereof. In an embodiment, the antibacterial agent and PhoU phosphatase activity inhibitor combination further include a pharmaceutically acceptable carrier.

The invention further includes kits. Kits include, for example, reagents for use in the methods of the invention, such as one or more plasmids including the coding sequence for one or more wild type or mutant phoU for example, for demonstration of defective production of persisters. The kit can include one or more bacteria including at least one mutation in a phoU gene.

### DEFINITIONS

An “agent” is understood herein to include a therapeutically active compound or a potentially therapeutic active compound. An agent can be a previously known or unknown compound. An agent can be selected or synthesized based on the known structure of PhoU, or may be part of a combinatorial library or a compound library of known and/or unknown chemical compounds. Agents can also be selected based on their ability to reduce PhoU activity or expression.

An “agonist” is understood herein as a chemical substance capable of initiating the same reaction or activity typically produced by the binding of an endogenous substance to its receptor. An “antagonist” is understood herein as a chemical substance capable of inhibiting the reaction or activity typically produced by the binding of an endogenous substance (e.g., an endogenous agonist) to its receptor to prevent signaling through a receptor or to prevent downstream signaling that is the normal result of activation of the receptor. The antagonist can bind directly to the receptor or can act through other proteins or factors required for signaling.

As used herein “amelioration” or “treatment” is understood as meaning to lessen or decrease the signs, symptoms, indications, or effects of a specific disease. For example, amelioration or treatment of a bacterial infection can include a reduction in bacterial load, especially reduction in persister bacterial load. As used herein, “prevention” is understood as to limit, reduce the rate or degree of onset, or inhibit the development of a disease or condition. Prevention can include maintaining a subject with a bacterial load less than can be detected, or less than can manifest signs or symptoms in a subject, or prevention of relapse. Prevention, amelioration, and treatment can be a continuum and need not be viewed as discrete activities. Prevention, amelioration, and treatment can be effected by one or more doses of an agent of the invention.

An “antibiotic” or “antibacterial agent” as used herein is understood as a compound inhibits or abolishes the growth of micro-organisms, such as bacteria, including bacteriostatic agents. Antibiotics include, for example, beta-lactams or cephalosporins, daptomycin, aminoglycosides, macrolides-lincosamides-streptogramins, linezolid, tetracylcines and quinolones, sulfa drugs or sulfonamides, piperazine, pyrantel pamoate. Beta-lactams include, for example the penicillins, cephalosporins, carbapenems and monobactams. Aminoglycosides include, for example, streptomycin, gentamicin, and neomycin. Macrolides, for example, azithromycin, clarithromycin, dirithromycin, erythromycin, roxithromycin, carbomycin A, josamycin, kitasamycin, midecamicine/midecamicine acetate, oleandomycin, spiramycin, troleandomycin, and tylosin/tylocine. Lincosamides include, for example, lincomycin and clindamycin. Streptogramins include, for example, pristinamycin and quinupristin/dalfopristin. Tetracyclines include, for example tetracycline, chlortetracycline, oxytetracycline, demeclocycline, doxycycline, lymecycline, meclocycline, methacycline, minocycline, rolitetracycline, tigecycline and glycylcycline antibiotics. Quinolones include, for example, cinoxacin, flumequine, nalidixic acid, oxolinic acid, piromidic acid, pipemidic acid, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin Mesilate, sparfloxacin, temafloxacin, tosufloxacin, clinafloxacin, gemifloxacin, moxifloxacin, gatifloxacin, sitafloxacin, trovafloxacin, ecinofloxacin, and prulifloxacin. Sulfa drugs or sulfonamides include, for example, acetazolamide, benzolamide, bumetanide, celecoxib, chlorthalidone, clopamide, dichlorphenamide, ethoxzolamide, indapamide, mafenide, mefruside, metolazone, probenecid, sulfacetamide, sulfadimethoxine, sulfanilamides, sulfamethoxazole, sulfasalazine, sultiame, sumatriptan, and xipamide.

A “cell free system” as used herein is a cell lysate that may or may not be fractionated. A cell free system can include purified proteins and nucleic acids.

As used herein, “changed as compared to a control reference sample” is understood as having a level of the analyte (e.g., colony forming unit) or activity (e.g., kinase activity, phosphatase activity) to be detected at a level that is statistically different than a sample from a normal, untreated, or control sample. Methods to select and test control samples was within the ability of those in the art. Depending on the method used for detection the amount and measurement of the change can vary. For example, a change in the amount of phosphorylation or dephosphorylation of analyte present will depend on the exact reaction conditions and the amount of time after exposure to the agent the sample is collected. Determination of statistical significance is within the ability of those skilled in the art.

As used herein, “colony forming unit” or “CFU” is understood as a bacteria capable of resulting in the growth of a single colony on a bacterial culture plate.

“Contacting a cell” or “contacting a bacterial cell” is understood herein as providing an agent to a bacterial cell, in culture or in an animal, such that the agent can interact with the surface of the cell, potentially be taken up by the cell, and have an effect on the cell. The agent can be delivered to the cell directly (e.g., by addition of the agent to culture medium or by application, e.g., topical application to an infected area), or by delivery to the organism by an enteral or parenteral route of administration for delivery to the cell by circulation, lymphatic, or other means.

As used herein, “detecting”, “detection” and the like are understood that an assay performed for identification of a specific analyte in a sample, or a product from a reporter construct in a sample. The amount of analyte detected in the sample can be none or below the level of detection of the assay or method.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug or a combination of drugs, a drug or combination of drugs which are “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

A combination of two or more agents can be prepared or provided in an effective dose. The combination of two drugs can be provided in as a mixed formulation (e.g., prepared for administration as a single dose as a single tablet, capsule, or vial) or packaged for co-administration (e.g., in a single blister pack, or otherwise packaged together). A combination of agents need not be administered simultaneously. It is understood that different compounds have different pharmacokinetic and pharmacodynamic properties which may suggest dosing on different schedules to maintain an effective dose of each of the agents. It is understood that an effective dose of the combination of agents may be in an amount that is less than the effective dose of one or both of the agents alone.

“Identify” or “identification” or the like as used herein as in “identification of an agent” is understood as characterization of a specific agent to determine specific characteristics of the agent to allow for determination of the chemical structures or properties of the agent. Identification can be accomplished by correlating the position, for example in the 96-well or 384-well plate to which the agent was added, or by determination of the chemical structure of an agent derived from a combinatorial chemistry library by NMR or other structural analysis, or by use of a radiofrequency tag or other identifying tag on the compound.

“Isoform” is understood herein as any of two or more functionally similar proteins that have a similar but not an identical amino acid sequence.

As used herein, “isolated” or “purified” when used in reference to a polypeptide means that a naturally polypeptide or protein has been removed from its normal physiological environment (e.g., protein isolated from plasma or tissue) or is synthesized in a non-natural environment (e.g., artificially synthesized in a heterologous system). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. “Isolated” when used in reference to a cell means the cell is in culture (i.e., not in an animal). Isolated cells can be further modified to include reporter constructs or be treated with various stimuli to modulate expression of a gene of interest.

As used herein “killing assay” is understood as an experiment to determine a change in the amount of viable bacteria or CFU per volume of bacteria (e.g., per ml) over time in response to exposing or contacting the bacteria with one or more agents sequentially and/or simultaneously. Cells can be at any phase of growth or in non-growing persister or dormant state during the assay. Assays can be performed at any temperature but typically at 37° C., with or without shaking in the case of liquid culture, or after exposure to the agents the viability of bacteria can be assessed by subculture in liquid medium or on solid medium.

“Kinase inhibitor” or “inhibitor of kinase activity” or the like as used herein is understood as an agent that reduces the phosphorylation activity, including autophosphorylation activity, of a kinase relative to the activity of the kinase under the same reaction conditions in the absence of the agent. For example, the activity of the kinase can be reduced by one or more agents by at least about 30%, at least about 40%, at least about 50%, at least about 60%, at least about 70%, at least about 80%, at least about 90%, at least about 95%, at least about 98%, at least about 99% relative to the reaction not including the agent. Similarly, a “phosphatase inhibitor” or “inhibitor of phosphatase activity” or the like as used herein is understood as an agent that reduces the dephosphorylation activity of a phosphatase relative to the activity of the phosphatase under the same reaction conditions in the absence of the agent. For example, the activity of the phosphatase can be reduced by at least one or more agents by at least about 30%, at least about 40%, at least about 50%, at least about 60%, at least about 70%, at least about 80%, at least about 90%, at least about 95%, at least about 98%, at least about 99% relative to the reaction not including the agent.

As used herein, “kits” are understood to contain at least the non-standard laboratory reagents for use in the methods of the invention, such as one or more plasmids including the coding sequence for one or more wild type or mutant phoU. The kit can include one or more bacteria including at least one mutation in a phoU gene. The kits can be used, for example, for screening libraries of compounds for inhibitors of PhoU phosphatase activity or for inhibitors of PhoU expression. Kits can also be used for the demonstration of lack of persister formation. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

The phrase “library of compounds” or “compound library” is understood as a plurality of chemical compounds that may or may not be related by one or more property, such as activity, e.g., kinase inhibitor, phosphatase inhibitor, metal chelator; structure, e.g., peptides, nucleic acids including antisense nucleic acids, carbohydrates, antibodies; products of combinatorial chemistry; or by approval status, e.g., FDA approved compounds for administration to humans. Groups of compounds with no obvious relation can also be considered a library.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. USA 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

“Obtaining” is understood herein as manufacturing, purchasing, or otherwise obtaining.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

A “PhoU containing bacteria” contain a “phoU gene” to allow for expression of a “PhoU protein.” PhoU containing bacteria and the like are understood as a bacteria that contains a gene and is capable of expressing a protein that has a sequence has at least about 20%, at least about 30%, at least about 40%, at least about 50%, at least about 60%, at least about 70%, at least about 80%, at least about 90% sequence homology/similarity to, preferably at least about 20%, at least about 30%, at least about 40%, at least about 50%, at least about 60%, at least about 70%, at least about 80%, at least about 90% identity to, at least one of the PhoU and/or PhoY1 and/or PhoY2 sequences provided herein (SEQ ID NOS: 1-5 and 16-19), particularly the E. coli PhoU sequence having Accession No. ZP_{—}00726232 (SEQ ID NO: 1) or an M. tuberculosis PhoY2 (SEQ ID NOS: 18 and 19). In an embodiment, the similarity or homology is throughout the length of the proteins, for at least about 200 amino acids of the protein, for at least about 150 amino acids of the protein, for at least about 100 amino acids of the protein, for at least 50 amino acids of the protein. A PhoU protein can be understood as a protein containing a PhoU domain or a PhoU domain sequence that has at least about 40%, at least about 50%, at least about 60%, at least about 70%, at least about 80%, at least about 90% sequence homology/similarity to, preferably at least about 40%, at least about 50%, at least about 60%, at least about 70%, at least about 80%, at least about 90% identity to, at least one of the PhoU domain sequences in FIG. 1G (SEQ ID NOS: 20-29). Further a PhoU protein has at least kinase or phosphatase activity.

Homology/similarity and identity can be readily determined using any of a number of publicly available sequence alignment tools including, but not limited to, BLAST (Basic Local Alignment Sequence Tool) available from the National Center for Biotechnology (NCBI) website at http://www.ncbi.nlm.nih.gov/blast/Blast.cgi or using ClustalW at the European Biology Labs (EBL) website at http://www.ebi.ac.uk/Tools/clustalw/index.html. Other tools to determine sequence homology and identity can be found at, for example, http://restools.sdsc.edu/biotools/biotoolsl.html. Sequence homology can also be determined by methods known to those in the art (e.g., see Taylor W R. J Mol Biol. 88:233-258, 1986). When not identified as being from a specific organism, e.g., E. coli PhoU or by use of a gene or protein name that is distinct e.g., PhoY1 or PhoY2 as a PhoU protein of M. tuberculosis, PhoU is understood to be a homolog that its at least about 20%, at least about 30%, at least about 40%, at least about 50%, at least about 60%, at least about 70%, at least about 80%, at least about 90% homologous to, preferably identical to, at least one of the PhoU and/or PhoY1 and/or PhoY2 sequences provided herein (SEQ ID NOS: 1-5 and 16-20), particularly the E. coli PhoU sequence having Accession No. ZP_{—}00726232 (SEQ ID NO: 1), or a gene encoding the same, and the protein has at least a kinase activity or a phosphatase activity. Other PhoU sequences are provided, for example, in SEQ ID NOS: 16-20. Kinase and phosphatase activity assays are described herein (e.g., see Examples herein). Such methods are known to those skilled in the art.

As used herein, a “PhoU specific antibody” preferentially binds a PhoU, more preferably a PhoU of a specific bacterial strain. Preferentially binds is having an affinity of at least 10³ fold, preferably at least 10⁴ fold, preferably at least 10⁵ fold higher than to non-specific protein.

As used herein, “plurality” is understood to mean more than one. For example, a plurality refers to at least two, three, four, five, one hundred, one thousand, or more.

“Reporter construct” as used herein is understood to be an exogenously inserted gene, often present on a plasmid, with a detectable gene sequence, under the control of a promoter sequence. The activity of the promoter is modulated upon binding of an agent that modulates transcription. Preferably, the gene product is easily detectable using a quantitative method. Common reporter genes include luciferase, beta-galactosidase, and green fluorescent protein (GFP). The reporter construct can be transiently inserted into the cell by transfection or transformation. Alternatively, stable cell lines or bacterial strains can be made by recombination using methods well known to those skilled in the art. The specific reporter gene or method of detection is not a limitation of the invention. The report construct comprised of phoU gene promoter fused to the above reporter genes when transfected or transformed into cells can be used to screen for compounds that inhibit the transcription of phoU gene expression.

A “sample” as used herein refers to a biological material that is isolated from its environment (e.g., blood or tissue from an animal; cells or conditioned media from a culture) and is suspected of, or contains an analyte, such as a product from a reporter construct or an active kinase or phosphatase. A sample can also be a partially purified fraction of a tissue or bodily fluid. A reference sample can be a “normal” sample, from a wild type bacteria or an uninfected subject or culture. A reference sample can also be from an untreated, but infected, subject sample or culture media; not treated with an active agent (e.g., no treatment or administration of vehicle only) and/or stimulus. A reference sample can also be taken at a “zero time point” prior to contacting the cell, culture, or subject with the agent to be tested.

A “subject” as used herein refers to living organisms. In certain embodiments, the living organism is an animal. In certain preferred embodiments, the subject is a mammal. In certain embodiments, the subject is a domesticated mammal. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of characteristics of the disease, condition, or syndrome such that a competent individual would diagnose or suspect that the subject was suffering from the disease, condition, or syndrome. Methods for identification of subjects suffering from or suspected of suffering from conditions such as bacterial infection is within the ability of those in the art. Subjects suffering from, and suspected of suffering from, a specific disease, condition, or syndrome are not necessarily two distinct groups.

“Therapeutically effective amount,” as used herein refers to an amount of an agent which is effective, upon single or multiple dose administration to the cell or subject, in prolonging the survivability of the subject or patient with such a disorder beyond that expected in the absence of such treatment.

An agent can be administered to a subject, either alone or in combination with one or more therapeutic agents, as a pharmaceutical composition in mixture with conventional excipient, e.g., pharmaceutically acceptable carrier.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1980). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### BRIEF DESCRIPTION OF THE FIGURES AND TABLES

FIGS. 1A-G. Cloning of E. coli phoU into an expression vector, confirmation of antibiotic resistance activity by complementation, and sequence alignments with PhoU homologs. (A) Schematic diagram of the pstSCAB-phoU operon and (B) the cloning site of the functional E. coli phoU gene into the TA cloning vector pCR®8/GW/TOPO. (C) Killing curve of log phase cultures of the PhoU mutant (JHU-313) and the wild type strain W3110 upon ampicillin treatment and (D) log phase cultures of the PhoU mutant transformed with the phoU gene and the vector control (JHU-313). (E) Alignment of PhoU protein sequences from Gram positive and Gram negative bacteria including E. coli (Accession No. ZP_{—}00726232 (SEQ ID NO: 1)); P. aeruginosa 01 (Accession No. YP_{—}001351460 (SEQ ID NO: 2)); S. aureus (Accession No. YP_{—}040800 (SEQ ID NO: 3); Mycobacterium bovis BCG str. Pasteur 1173P2 PhoY1 (Accession No. YP_{—}979414 (SEQ ID NO: 4); and Mycobacterium bovis BCG str. Pasteur 1173P2 (Accession No. YP_{—}976968 (SEQ ID NO. 5)) is shown. PhoY1 and PhoY2 sequences from Mycobacterium tuberculosis H37Rv are provided in the Sequence listing as SEQ ID NOS: 16-19. (F) PhoU motifs indicated schematically on three protein sequences. (G) Alignment of PhoU motif sequences from 1T72_D, Aquifex Aeolicus (SEQ ID NO: 20); gi 13632816, Caulobacter vibrioides (SEQ ID NO: 21); gi 7388010, Sinorhizobium meliloti (SEQ ID NO: 22); gi 10720228, Zymomonas mobilis (SEQ ID NO: 23); 1SUM_B, Thermotoga maritime (SEQ ID NO: 24); gi 10720214, Burkholderia sp. (SEQ ID NO: 25); gi 61229895, Streptococcus pneumoniae (SEQ ID NO:26); 1VCT_A, Pyrococcus horikoshii (SEQ ID NO: 27); gi 74510295, Methanothermobacter thermautotrophicus (SEQ ID NO: 28); and 1 SUM_B, Thermotoga maritime (SEQ ID NO: 29).

FIGS. 2A-C. Survival of the PhoU mutant (▪) and wild type E. coli strain W3110 (♦) to antibiotic exposure over time. (A) A killing curve of log phase bacteria exposed to ampicillin 100 μg/ml in LB medium. (B) A killing curve of overnight stationary phase cultures exposed to ampicillin 100 μg/ml in LB medium. (C) A killing curve of overnight stationary phase cultures exposed to norfloxacin 3 μg/ml in LB medium. The viability was measured by determination of CFU. Dashed line indicates JHU-313, solid line, W3110. (Note different scales.)

FIGS. 3A-C. Susceptibility of the PhoU mutant JHU-313 and wild type E.

coli wild type strain W3110 to a variety of stresses. (A) A killing curve demonstrating susceptibility to starvation in saline. Dashed line indicates the PhoU mutant JHU-313, solid line, W3110. (B) A killing curve demonstrating susceptibility to acid pH 4.0 in LB. Wild type W3110, solid line with ♦; JHU-313, dashed line with ▴; JHU-313 with empty plasmid vector; dashed line with ▪; JHU-313 with phoU plasmid containing vector, solid line with M. (C) A killing curve demonstrating susceptibility to energy inhibitors, 1 mM CCCP and 5 mM DCCD in pH 5.0 MOPS minimal medium. Dashed line indicates the PhoU mutant JHU-313, solid line, W3110. (Note different scales.)

FIGS. 4A-B. Susceptibility of E. coli PhoU mutant strain to tuberculosis drug PZA. (A) A killing curve demonstrating susceptibility of the PhoU mutant JHU-313 and its complemented strain and wild type strain W3110 to TB persister drug PZA at pH5.0 in MOPS minimal medium. (B) Comparison of log phase and stationary phase cultures of the PhoU mutant JHU-313 and the wild type strain W3110 to PZA (2 mg/ml) exposure in MOPS minimal medium (pH5.0) over time. Dotted line represents the PhoU mutant and solid line wild type strain W3110.

FIG. 5. Western blot analysis of PhoU expression in E. coli wild type strain W3110 in response to nutrient availability. Lane 1, 27 kD molecular weight marker; Lane 2, W3110 grown overnight in MOPS minimal medium with 2 mM K₂HPO₄; Lane 3, log phase growth of W3110 grown in rich medium LB medium; Lane 4, stationary phase growth of W3110 grown in LB medium.

FIG. 6. Kinase activity of PhoU. (A) E. coli PhoU, PhoB, and PhoR were recombinantly expressed and purified. Purified proteins were incubated with [γ³²P]-ATP under conditions to permit phosphorylation. Reactions were resolved by SDS-PAGE. (A) Stained gel and (B) autoradiograph to reveal phosphorylation. Arrow in (B) indicates phosphorylated PhoU.

FIG. 7. Phosphatase activity of E. coli PhoU. Phosphatase activity of PhoU was tested using the EnzChek phosphatase assay kit, and the reaction was monitored for a change in fluorescence over time.

FIG. 8. Effect of cations on E. coli phophatase activity. Effect of Fe³⁺, Fe²⁺, Mg²⁺, Zn²⁺, Ca²⁺, and Mn²⁺ on phosphatase activity was tested using the EnzChek assay kit.

FIGS. 9A-B. Effect of Fe³⁺ and inorganic phosphate (K₂HPO₄) on wild type and mutant E. coli PhoU phosphatase activity. Phosphatase activity of wild type E. coli PhoU protein and mutant E. coli PhoU protein, PhoU118 and PhoUG219H, was tested in the presence of (A) Fe3+ and (B) inorganic phosphate using the EnzChek assay kit.

FIGS. 10A-B. Phosphatase activity of M. tuberculosis PhoU homologs PhoY1 and PhoY2. Phosphatase activity of (A) PhoY1 and (B) PhoY2 in the presence of Fe²⁺, Fe³⁺, and inorganic phosphate as indicated.

FIG. 11. Inhibition of phosphatase activity of PhoY2 using known compounds. The effect of a series of FDA pharmaceutically acceptable compounds were tested for the ability to inhibit PhoY2 phosphatase activity. Bars in each group left to right are the order of the compounds listed in the legend.

Table 1 shows MIC/MBC values (μg/ml) for the wild type strain of W3110 and the PhoU mutant (JHU-313), the PhoU mutant transformed with an expression vector including the PhoU sequence (JHU-313 containing PhoU), and the PhoU mutant transformed with an empty expression vector (JHU-313 containing pVector). MIC and MBC values were determined by using serial two-fold dilutions.

Table 2 shows persister specificity of the wild type strain W3110 and the PhoU mutant upon sequential exposure to different antibiotic agents. Bacterial cultures were grown in LB broth to log phase when they were exposed to ampicillin at 100 μg/ml. After exposure to ampicillin for 1.5 hours, cells were washed by centrifugation and resuspended in LB containing gentamicin (20 μg/ml), trimethoprim (16 μg/ml), norfloxacin (5 μg/ml), and again ampicillin (100 μg/ml) and incubated for an additional 20 hours. Colony forming units (CFU) were determine at the start, after 1.5 hours ampicillin treatment, and at 20 hours after second antibiotic exposure.

Table 3 shows genes in the PhoU mutant JHU-313 that were upregulated at least two-fold compared to the wild type strain W3110 in DNA microarray analysis. Bacteria used for RNA isolation were grown in MOPS minimal media supplemented with 0.4% glucose and 2 mM K₂HPO₄ at 37° C. MasterPure RNA Purification Kit was used for RNA isolation following manufacturer's instructions. Affymetrix E. coli gene chips (triplicate arrays for both wild type and the PhoU mutant strain) were used in the DNA microarray experiment. Selected genes with known functions from a list of about 350 genes up-regulated at least two fold were grouped according to function as shown in the table. Expression of phoA, fliA, purK, and sgrS demonstrated at least a five-fold change in expression, and phoE demonstrated at least a 10-fold change in expression.

Table 4 shows the sensitivity of the PhoU mutant and the complemented strain to antibiotics and peroxide as measured by zone inhibition (mm). The sensitivity of the bacterial strains to antibiotics or peroxide was determined by Kirby-Bauer's paper disc assay as measured by zone inhibition in millimeters.

Table 5 shows a comparison of log phase and stationary phase cultures of the wild type strain of W3110, the PhoU transposon mutant (JHU-313), and the PhoU deletion mutant (ΔphoU) susceptibility to ampicillin at 100 μg/ml (Ap100) and norfloxacin at 3 μg/ml (Norf3). The CFU values were determined at different times of exposure to antibiotics for both log phase cultures and stationary phase cultures as indicated. The ΔphoU mutant was generated using methods known in the art.

Tables 6A-B show MIC and MBC values for wild type and mutant, PhoY1, and wild type and mutant PhoY2 upon exposure to PZA, INH, and RIF (A) on plates and (B) in liquid culture.

Table 7 shows results from a killing assay of wild type and mutant PhoY1, and wild type and mutant PhoY2 in response to PZA, RIG, and ADC at acidic or neutral pH.

Table 8 shows the kinetic properties of wild type and mutant E. coli PhoU.

Table 9 shows results from a killing assay for wild type W3110, mutant W3110ΔphoU, or mutant W3110ΔphoU transformed with expression vectors carrying wild type, mutant, or truncated versions of E. coli phoU. Cells were grown in the presence of low or high inorganic phosphate (Pᵢ), Fe³⁺, Fe³⁺ with low inorganic phosphate, Fe³⁺ with high inorganic phosphate.

Table 10 shows the increased killing effect of M. tuberculosis therapeutics in combination with PhoY2 phosphatase inhibitors in various combinations.

### DETAILED DESCRIPTION

The invention includes the discovery that persister formation in bacteria requires the activity of a PhoU protein, particularly the phosphatase activity of a PhoU protein. Disruption of PhoU function by mutation or deletion of PhoU from the bacteria prevents or greatly reduces persister formation. Similarly, disruption of PhoU phosphatase activity by mutation or use of a chemical phosphatase activity prevents persister formation. Similarly antisense nucleic acids and antibodies can also be used to disrupt PhoU activity and reduce persister formation.

The invention includes a method to identify agents to inhibit persister formation by identifying agents that inhibit phosphatase activity of a PhoU protein. Phosphatase activity can be tested, for example, using commercially available phosphatase assays such as EnzChek phosphatase kit from Molecular Probes (Invitrogen). Phosphatase activity can also be tested, for example, by performance of a killing assay in bacteria. PhoU expressing bacteria and/or plasmids for expression of wild type and mutant PhoU proteins can be sold in a kit. Such kits can be used, for example, for identifying a PhoU phosphatase inhibitor.

The invention includes the use of one or more phosphatase inhibitors for the prevention of persister formation in a subject, particularly a patient. The phosphatase inhibitor can be used alone or in combination with other phosphatase inhibitors. The phosphatase inhibitor(s) can also be used in combination with one or more antibiotic agents for the prevention, amelioration, or treatment of a subject susceptible to, suspected of having, or known to have a bacterial infection, particularly an infection with a bacterial infection with a bacteria known to undergo persister formation.

The invention includes pharmaceutical compositions containing a phosphatase inhibitor preferably in combination with an antibacterial agent or antibiotic, optionally further including one or more pharmaceutically acceptable carrier. The invention includes the use of an antibacterial agent in combination with a phosphatase inhibitor, optionally further including one or more pharmaceutically acceptable carrier for the prevention, amelioration, and/or treatment of a bacterial infection, particularly with a bacteria known to undergo persister formation.

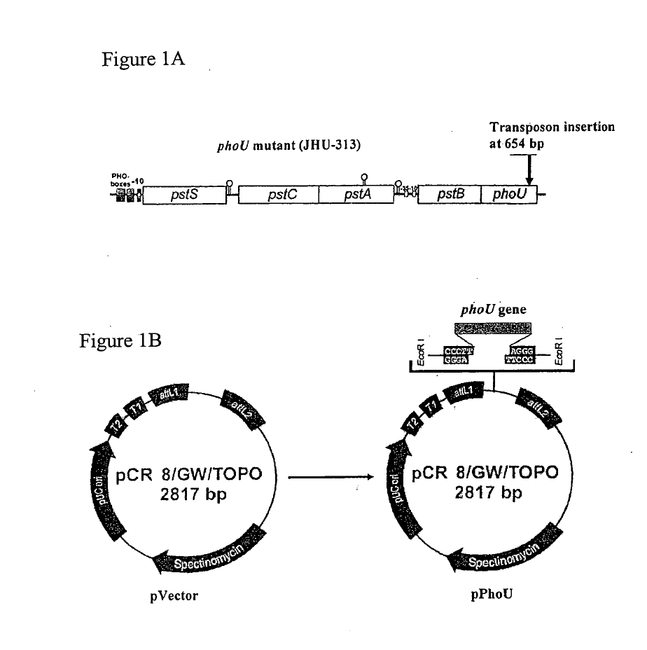

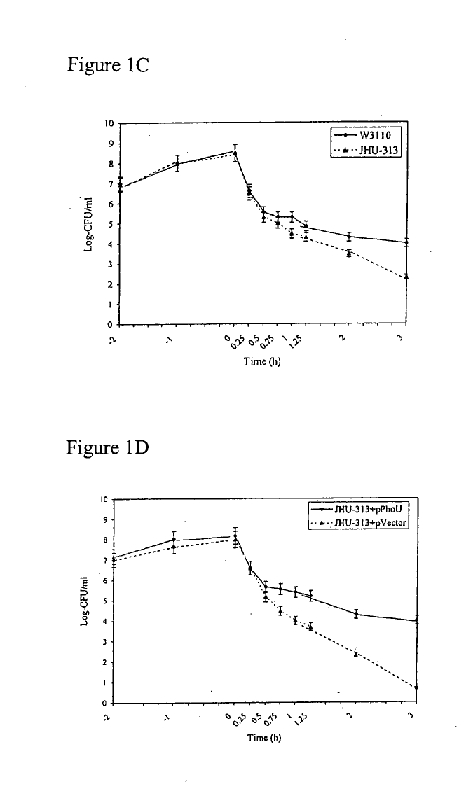

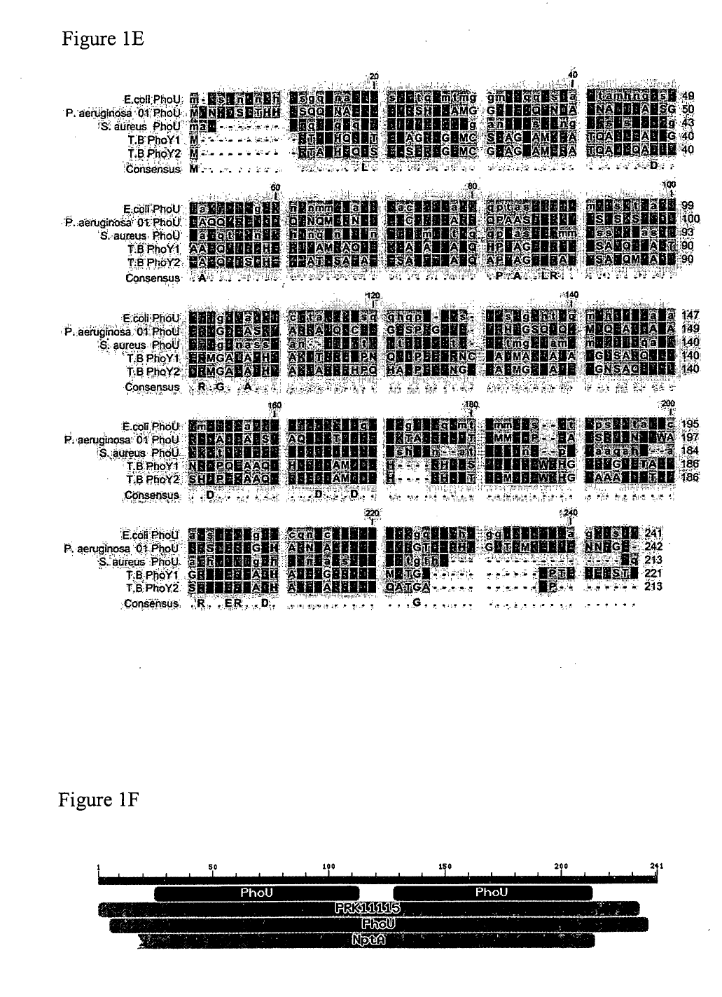

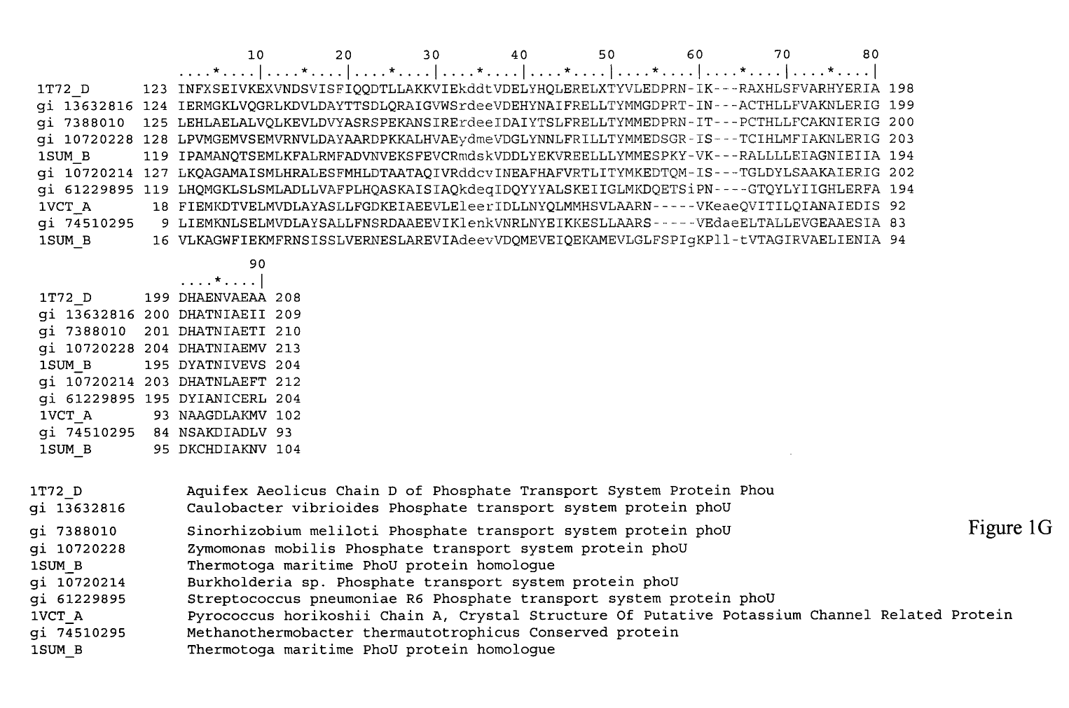

Persisters are known to be tolerant to multiple antibiotics and stresses (10, 11, 13, 22, 27). Disclosed herein, a persister gene phoU involved in persister formation has been identified (FIG. 1). The inactivation of phoU leads to increased susceptibility to multiple antibiotics and stresses. The various PhoU phenotypes disclosed herein are generally more obvious in stationary phase or starved cultures than in log phase cultures when compared with the wild type strain W3110. This is presumably because persister formation in stationary phase is defective in the PhoU mutant; therefore, the stationary phase culture without persisters will die off sooner. In contrast, persister formation in the wild type stationary phase cultures with functional PhoU is normal. This leads more persisters to form, and thus to be more obvious difference in susceptibility to antibiotic or stress treatments the PhoU mutant.

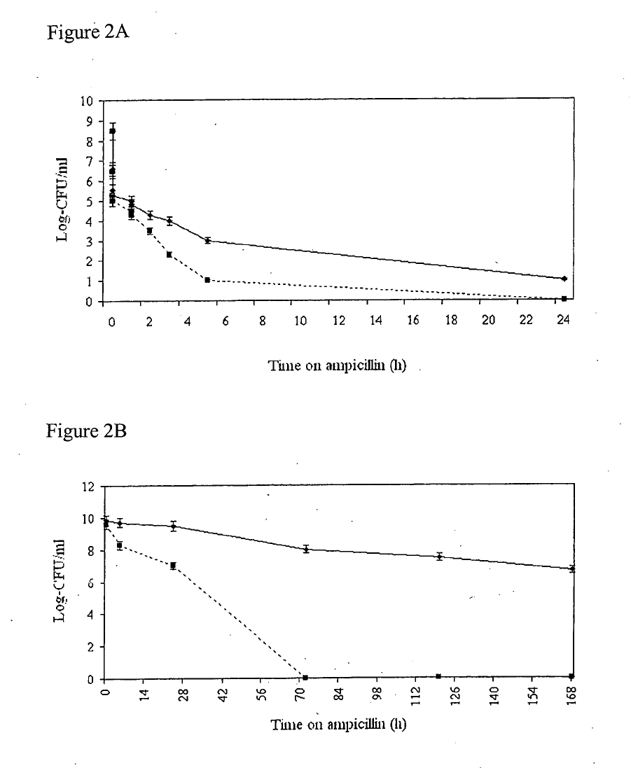

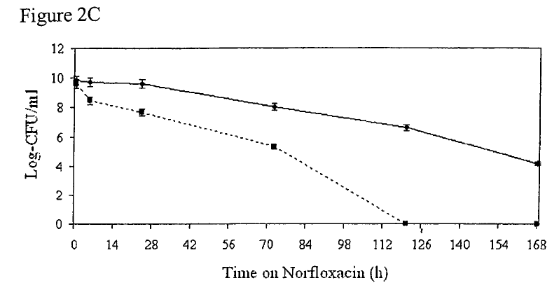

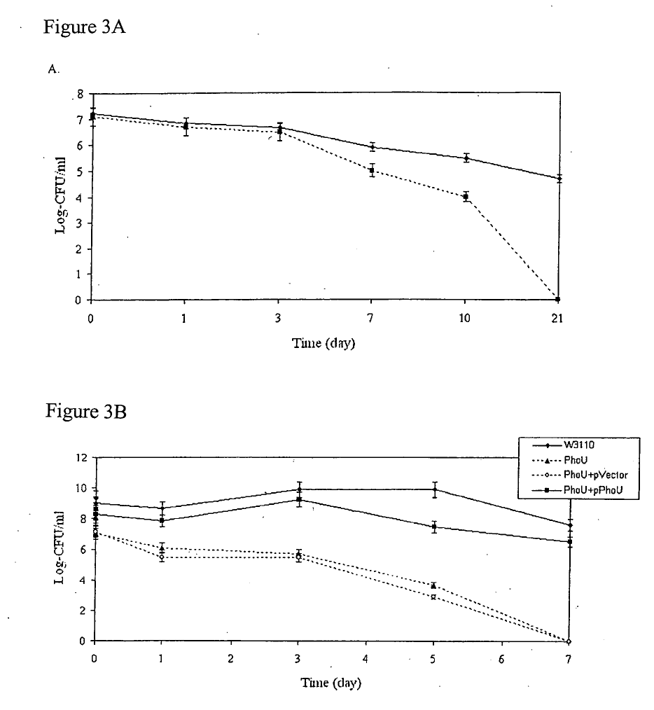

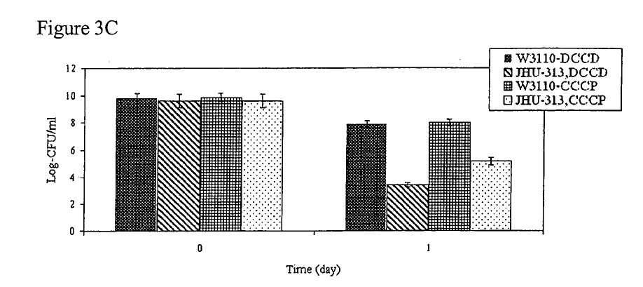

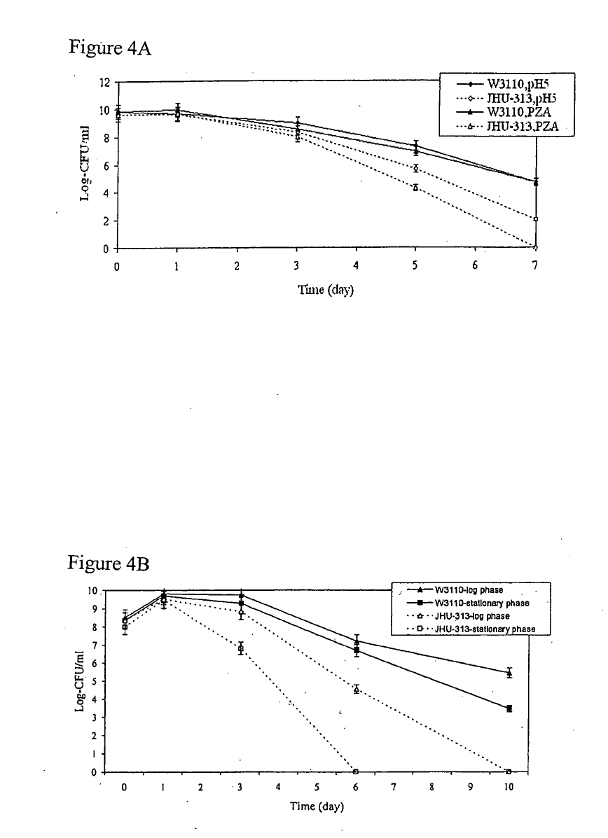

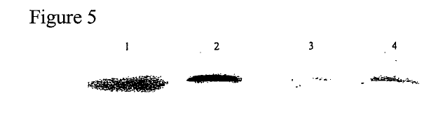

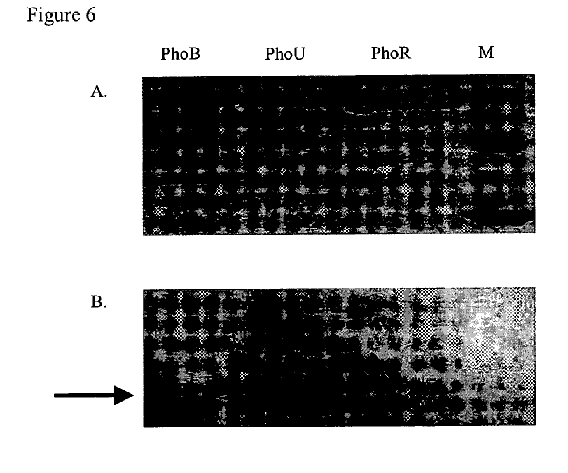

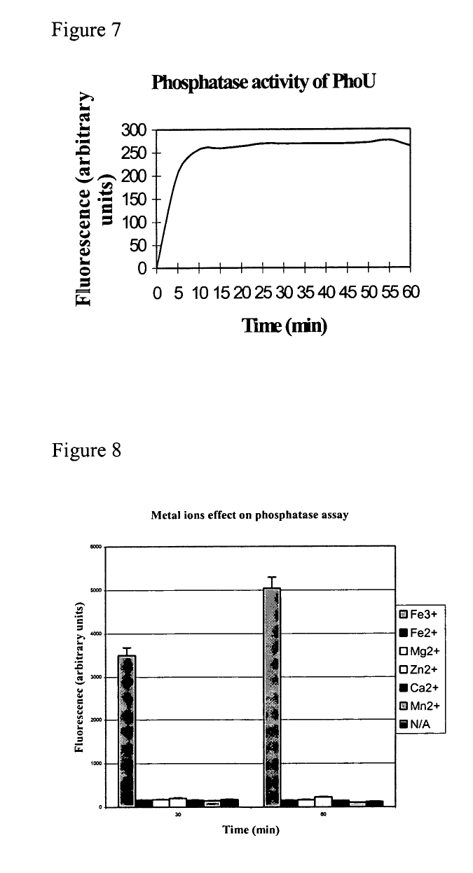

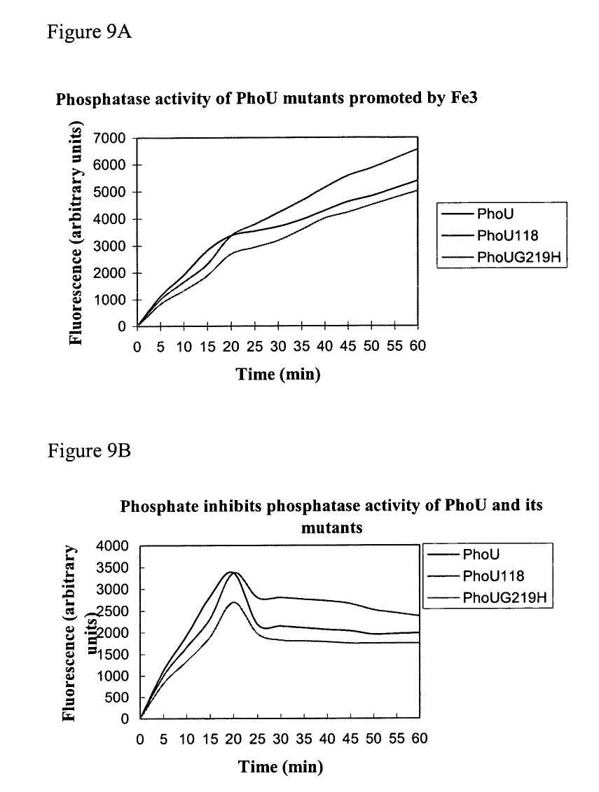

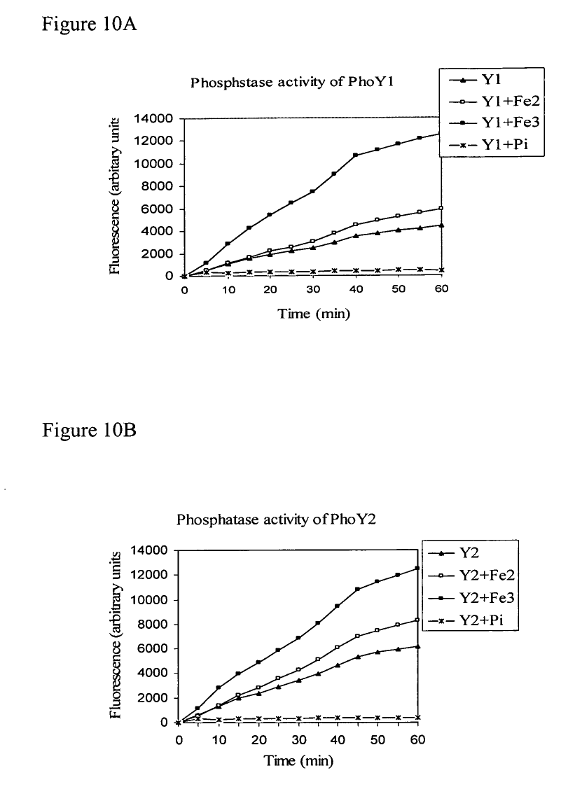

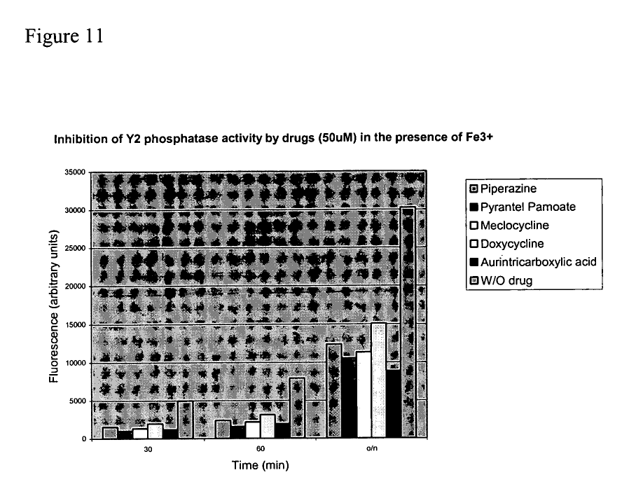

It should be emphasized that “persisters” are relative and should be defined by highly specific conditions such as the type of antibiotics, antibiotic concentrations, the length of antibiotic exposure, the culture media and the growth phase. Thus during short term antibiotic exposure when the PhoU mutant is not completely killed there can be a specific “persister” frequency (FIG. 1C). However, in longer antibiotic or stress exposure when there are no viable bacteria left as found in the PhoU mutant (FIG. 2B, FIG. 3B), persister frequency has no meaning as no persister frequency can be determined. This is an important observation that suggests that “persisters” are not homogeneous, and consist of different bacterial subpopulations that are defined by specific conditions and times as discussed herein.

PhoU was originally identified as a specific negative regulator for the Pho regulon (B. L. Wanner, In F. C. Neidhart at al. (ed.), Escherichia coli and Salmonella: cellular and molecular biology, 2nd ed. p. 1357-1381. American Society for Microbiology, Washington, D.C. (1996)). However, the findings disclosed herein include the discovery that that the PhoU mutant has a diverse phenotype. Incativation of PhoU results in a substantial increase in susceptibility to various antibiotics tested (ampicillin, norfloxacin, gentamicin, tetracycline, trimethoprim) and stress conditions (starvation, heat, peroxide, acid pH, weak acids, energy inhibitors). Array data demonstrate paradoxically higher metabolic activity as demonstrated by increased expression of flagella synthesis genes and energy production genes (Table 3) strongly suggest that the function of PhoU is beyond its role in phosphate metabolism and serves as a global negative regulator that facilitates persister formation.

Based on the multiple phenotypes of PhoU beyond its original role in phosphate metabolism and in particular its association with persisters it is proposed that the gene phoU be renamed as persister formation gene, perF, to more accurately reflect the diverse functions of this protein. This study provides the first evidence that PhoU is a master regulator involved in persister formation, whose inactivation leads to loss of persisters as the underlying mechanism for the increased sensitivity to antibiotics and stresses.

Previously, it has been shown that pstSCAB-phoU operon expression manifests the interesting property of “phase variation” as demonstrated by switching on-and-off of the Lac+ and Lac− phenotype mediated by phoA-lacZ controlled by the pst-phoU in response to diverse environmental changes, such as the type of media (rich medium versus minimal medium), the presence of carbon source, and the age of bacteria (B. L. Wanner, J. Mol. Biol. 191, 39 (1986)). Based on the findings disclosed herein on the role of PhoU in persister formation and the effect of nutrient availability on PhoU expression (FIG. 4B), phosphatase activity (FIGS. 9 and 10), and the “phase variation” property of the pstSCAB-phoU operon, a model for persister formation is proposed. Although not wishing to be bound by mechanism, a persister formation with PhoU as a master switch is suggested to occur as follows: When bacteria are growing in the presence of sufficient nutrients (including phosphate) as in rich medium such as LB medium, PhoU, as a negative regulator for cellular metabolism, is repressed or not expressed in the majority of the bacterial population (FIG. 4B). This reduced PhoU expression makes them susceptible to antibiotics and stresses. However, a small number of bacteria express low amount of PhoU because of incomplete repression of the pstSCAB-phoU operon due to “phase variation,” presumably caused by competing transcription activators and repressors in the promoter region of this operon. This causes a low level oscillatory or rhythmic transcription of the pstSCAB-phoU operon in response to changes in fluctuating environments. This allows persister formation in a small number of bacteria even during log phase growth. However, as bacteria enter stationary phase or encounter nutrient starvation, including phosphate starvation, PhoU is induced and expressed to higher level (FIG. 4B). This allows more persisters to form. The function of PhoU is to serve as a negative global regulator, which suppresses the overall cellular metabolic activity of the bacteria through affecting the genes or proteins involved in energy production and membrane transporters, to allow persister formation.

The current persister model based on toxin-antitoxin modules is challenged by the recent observation that overexpression of unrelated toxic proteins, such as DnaJ, also increased persister formation. The persister model presented herein based on PhoU and the M. tuberculosis homolog PhoY2 (which is widely present in both Gram-negative and Gram-positive bacteria) which explains the pleiotropic phenotype of persisters that exhibit tolerance to various antibiotics and stresses. It also provides an explanation of the stochastic nature of persister generation in response to fluctuating environmental changes and is consistent with the proposal that persisters represent specialized survivor cells whose production is regulated by the growth stage of the bacterial population. Since PhoU is present in many bacterial genomes, but not in eukaryotic genomes, PhoU is likely to be involved in persistence in other bacterial species.

Persister bacteria pose enormous public health problems. The persister tubercle bacilli (TB) present a tremendous challenge for effective TB control and underlie the lengthy TB therapy. This makes patient compliance very difficult and is in part responsible for the increasing emergence of drug resistant TB such as the recently reported extreme drug resistant TB (XDR-TB) (J. Cohen, Science 313, 1554 (2006)). The finding that PhoU is a persister switch has implications for design of new drugs that target persister bacteria and will result in improved therapeutics and treatment of many persistent bacterial infections such as that caused by M. tuberculosis.

### Example 1

### Culture Media, Antibiotics, and Chemicals

Luria-Bertani (LB) broth or agar was used as the growth medium for most experiments. MOPS (morpholinepropanesulfonic acid) minimal medium or M9 minimal medium was used a nutrient-deficient medium. Glucose was added as a carbon source to a final concentration of 0.4%. Saline (0.9% NaCl) was used in the starvation experiment. The antibiotics ampicillin, norfioxacin. gentamicin. trimethoprim, and kanamycin and stress agents hydrogen peroxide, carbonyl cyanide m-chlorophenylhydrazone (CCCP), salicylic acid, pyrazinoic acid, and pyrazinamide (PZA) were obtained by Sigma Chemical Co., and their stocks were dissolved in appropriate solvents and used at appropriate concentrations as indicated below.

### Example 2

### Bacterial Strains, Construction of Mutant Library and Library Screen, DNA Manipulations, Inverse PCR, and DNA Sequencing

E. coli K-12 W3110 is F⁻ mcrAmcrB IN(rrnD-rrnE) I lambda⁻. Bacteriophage λ NK1316, containing TnI0 kan c1857 Pam80 nin5 b522 att-, was used for the construction of the E. coli transposon mutant library. Wild-type E. coli K-12 strain W3110 was subjected to mini-Tn10 (kanamycin) transposon mutagenesis using a method described previously (Falla et al, 1998). The mutant library consisting of 11,748 clones was grown in LB medium containing 50 μg/ml kanamycin in 384-well plates overnight. The library in 384-well plates was replica transferred to fresh LB medium in 384-well plates, which were incubated at 37° C. for 5 h to log phase when ampicillin was added to 100 μg/ml. The plates were further incubated for 24 h when the library was replica transferred to LB plates to score for clones that failed to grow after ampicillin exposure.

### Example 3

### Identification of a Persister Gene phoU by Mutant Library Screen

In the previous study that identified the persistence gene hipA, the screen was based on identifying mutants that had increased persistence or survival upon antibiotic exposure compared with the parental strain. To better understand the mechanism of persisters and to identify new genes involved in persister formation, a different genetic screen was performed to identify potential mutants with decreased persistence in E. coli using mini-TnI0 transposon mutagenesis (N. Kleckner, J. Bender, S. Gottesman, Methods Enzymol. 204, 139 (1991), incorporated herein by reference). The persistence defective mutant screen identified several mutants that failed to grow on LB plates after ampicillin exposure.

One mutant JHU-313 that consistently gave the phenotype of inability to grow upon subculture after ampicillin exposure was further characterized. Sequence analysis revealed that this mutant harbored a transposon insertion near the C-terminus at 654 by of the phoU gene (FIG. 1A), which encodes a negative regulator for phosphate metabolism. Homology search revealed that PhoU is present in numerous bacterial species, both Gram positive and Gram negative (e.g., see FIG. 1E). It is interesting to note that M. tuberculosis, which is notorious for its persistence, has two PhoU homologs, PhoY2 and PhoY1, in its genome (S. T. Cole et al., Nature 393, 537 (1998)).

### Example 4

### Identification of Transposon Insertion Site and Plasmid Construction

Inverse PCR was used to localize the mini-TnI0 insertions in mutant E. coli. Two oligonucleotide primers at the end of IS903 of the mini-TnI0 derivative 103 (11) were synthesized (primer 1,5′-TTA CAC TGA TGA ATG TTC CG-3′ (SEQ ID NO: 6), and primer II, 5′-GTC AGC CTG AAT ACG CGT-3′(SEQ ID NO: 7)). Chromosomal DNA of mutant strains was isolated and digested by the restriction enzyme HaeII or AvaII, and DNA restriction fragments were then circularized using T4 DNA ligase (Invitrogen). The PCR cycling parameters were 1 min at 96° C. followed by 30 cycles, each consisting of 10 s at 96° C., 30 s at 55° C., and 2 min at 65° C. PCR products were subjected to DNA sequencing with primer I as the sequencing primer. The DNA sequences of the PCR products were subjected to a homology search in the NCBI database using the BLAST algorithm.

The primers used for the construction of the plasmid containing a functional phoU gene are F (5′-CGC ATA TGT TAT GTA CCT GGG CGA ATT G-3′ (SEQ ID NO: 8)) and R (5′-CCG GAT CCT CAT TAT TTG TCG CTA TCT TTC C-3′ (SEQ ID NO: 9)). The purified PCR product was cloned using a pCR8/GW/TOPO TA cloning kit (Invitrogen, Carlsbad, Calif.) according to the manufacturer's protocol. The plasmid construct containing the phoU gene and a vector control were used to transform the PhoU mutant by electroporation. The deletion mutants of phoR, phoB, phoU, hipA, and hipAB were constructed as described previously by Datsenko and Wanner (7), incorporated herein by reference.

### Example 5

### Reduced Persister Formation in PhoU Disrupted E. coli is Complemented by Plasmid Expression of PhoU

To determine the kinetics of killing by ampicillin (100 μg/ml), a killing curve experiment was performed comparing the PhoU mutant and the wild type strain W3110 over time for log phase cultures (FIG. 1C). For log phase cultures, the PhoU mutant was initially killed as much as the wild type during the first 0.5-1 hr, but showed increased susceptibility to ampicillin and by 3 hr the PhoU mutant was killed about 100-fold more than the wild type strain.

Complementation of the PhoU mutant with the functional phoU gene restored the level of antibiotic susceptibility to that of the wild type strain, whereas the PhoU mutant transformed with vector control remained susceptible to ampicillin (FIG. 1D). These data demonstrate that the transposon mutant can be fully complemented by the expression of PhoU from a plasmid.

### Example 6

### Reduced Persister Formation is More Obvious in Stationary Phase Cultures Than Log Phase Cultures

A killing curve experiment was performed on W3110 and JHU-313 bacteria growing both at stationary and log phase.

For log phase cultures, no viable cells remained in the PhoU mutant culture, whereas the wild type still had 10-100 viable bacteria left after 24 hr exposure to ampicillin at the end of the experiment (FIG. 2A). A more dramatic difference between the PhoU mutant and the wild type strain in susceptibility to ampicillin (100 μg/ml) exposure was seen for stationary phase cultures (FIG. 2B). It is well known that stationary phase cultures are not susceptible to ampicillin or penicillin (G. L. Hobby, K. Meyer, E. Chaffee, Proc. Soc. Exp. Biol. NY 50, 281 (1942)).

Surprisingly, the stationary phase PhoU mutant was completely sterilized by ampicillin after 72 hr. The wild type strain showed the typical high tolerance to ampicillin with only a slight drop in viable cells with 10⁸ CFU/ml (FIG. 2B). Similarly, the stationary phase PhoU mutant was also highly susceptible to the quinolone drug norfloxacin (3 μg/ml) compared with the wild type strain (FIG. 2C), such that no viable bacteria were left in the PhoU mutant after 120 hr exposure while the wild type strain had about 10⁷ CFU/ml left at the same exposure time (FIG. 2C).

### Example 7

### PhoU Mutant is More Susceptible to Various Antibiotics of Diverse Structures

Susceptibility of PhoU mutant to various antibiotics was tested. Minimum inhibitory concentrations (MICs) and minimum bactericidal concentrations (MBCs) were determined by using serial twofold dilutions of the antibiotics in LB broth. The initial cell densities were 10⁶ to 10⁷ bacteria/ml of log phase cultures, and the samples were incubated for 16 h at 37° C. The susceptibilities of the log phase and stationary-phase PhoU mutant JHU-313 and wild type W3110 cultures to various antibiotics, including ampicillin (100 μg/ml), norfloxacin (3 μg/ml), gentamicin (20 μg/ml), trimethoprim (16 μg/ml), and PZA (2 mg/ml), were evaluated in a drug exposure experiment in MOPS minimal medium (pH 5.0). The antibiotic exposure was carried out over a period of several hours to 10 days at 37° C. without shaking.

Aliquots of bacterial cultures exposed to antibiotics were taken at different time points and washed in saline before plating for viable bacteria (CFU) on LB plates. The MIC was recorded as the minimum drug concentration that prevented visible growth, and the MBC was recorded as the drug concentration that reduced CFU by 100-fold over the seeded inoculum.

Interestingly, the PhoU mutant was found to be more susceptible to all the antibiotics tested than the wild type strain W3110 in both minimum inhibitory concentration (MIC) and minimum bactericidal concentration (MBC) type of experiments (Table 1A). In the MIC and MBC type of experiments, the PhoU mutant was generally 2-10 fold more susceptible to various antibiotics than the wild type (Table 1A). To determine if the PhoU mutant can be complemented by the phoU gene, the JHU-313 PhoU mutant was transformed with an expression vector containing a functional phoU gene or an empty expression vector (FIG. 1B). Transformation of the PhoU mutant with the phoU gene conferred increased resistance to antibiotics to the level of the wild type (Table 1). These data demonstrate that the PhoU is necessary for persister formation in E. coli.

### Example 8

### Persister Formation Results in Broad Antibiotic Resistance

To determine whether the persister bacteria after ampicillin treatment are susceptible to other antibiotics, the persister bacteria of wild type strain W3110 and the PhoU mutant from log phase culture pre-exposed to ampicillin (100 μg/ml) for 1.5 hr were exposed to gentamicin (20 μg/ml), trimethoprim (16 μg/ml), norfioxacin (5 μg/ml) and also ampicillin again in LB broth, and incubated for 20 hr before CFU determination. Both wild type and the PhoU mutant had a 3-log decrease in CFU count with comparable number of persisters left after 1.5 hr ampicillin treatment (Table 2).

Susceptibility of PhoU mutant to various stresses. Overnight cultures of the PhoU mutant and the wild-type strain W3110 grown in LB broth at 37° C. were incubated with acid, pH 4, at 37° C. at 58° C., respectively, and incubated for various times, and the number of CFU per millimeter was determined by plating serial dilutions of cells on LB plates. For carbon starvation, cultures were grown overnight in M9 minimal medium with 0.4% glucose and then washed twice with saline. The cultures were diluted 1:100 in saline and incubated without shaking at 37° C. at different time points. The susceptibilities of the PhoU mutant and the wild-type strain W3110 to weak acids were tested by incorporating salicylate (80 μg/ml) and pyrazinoic acid (230 μg/ml) into LB agar with acid at pH 5.0 in an MIC experiment wherein the growth inhibition was assessed by visible growth after incubating the LB plates at 37° C. overnight.

Interestingly, the persisters in the wild type W3110 bacteria not killed by ampicillin treatment remained tolerant to ampicillin, and were also non-susceptible to other antibiotics with comparable number of persisters. This indicates that the persisters are multidrug tolerant, a finding consistent with previous observations (C. Wiuff at al., Antimicrob. Agents Chemother 49, 1483 (2005)). In contrast, unlike the wild type strain, “persisters” not killed by the short ampicillin exposure (1.5 hr) in the PhoU mutant continued to be killed by bactericidal antibiotics ampicillin, norfloxacin, and gentamicin, but interestingly not by the bacteriostatic antibiotic trimethoprim (Table 2).

### Example 9

### PhoU Mutant is More Susceptible to a Variety of Stresses Including Starvation, Heat, Oxidative Stress, Acid pH, Weak Acids, and Energy Inhibitors

The sensitivity of bacterial strains to antibiotics or stresses was also assessed by the Kirby-Bauer method (2) using paper discs. E. coli bacteria were grown to log phase (10⁸ bacteria) in LB broth. An inoculum from this culture was spread across the surfaces of LB plates to provide confluent growth. Nitrocellulose discs (7 mm in diameter) soaked with appropriate antibiotics or stress agents (100 mM H₂O₂, were placed on the agar surface). After incubation at 37° C. for 48 h, the diameter of the zone of growth inhibition was measured and scored according to the size of the zone of inhibition, which is directly proportional to the sensitivity of the organism to the antibiotic. The results obtained were reproducible.

To determine the effect of starvation on survival of the PhoU mutant, the PhoU mutant and the wild type strain W3110 grown in M9 minimal medium, were subsequently subjected to starvation in saline for various times and assessed their ability to survive starvation. During the first 3 days of starvation, there was no apparent difference between the PhoU mutant and the wild type strain (FIG. 3A). However, more pronounced susceptibility to starvation of the PhoU mutant was seen after 1 week of starvation. No surviving bacteria were detected in the PhoU mutant after 3 weeks of starvation, whereas the wild type had 3×10⁴ viable bacteria (FIG. 3A). This indicates the PhoU mutant was more sensitive to starvation than the wild type strain.

The PhoU mutant was much more sensitive to heat treatment as demonstrated by no survivors after 2 hr exposure at 58° C. for both log phase and stationary phase cultures, whereas the wild type strain W3110 had 3×10³ and 1.7×. 10⁴ surviving bacteria from the initial culture of 10⁸ CFU/ml for the log phase and stationary phase cultures, respectively. The PhoU mutant was also tested for its ability to grow at 42° C. While wild type strain W3110 grew normally at 42° C. on LB plates, whereas the PhoU mutant grew very poorly (not shown).

In acid pH4.0 exposure experiment, the PhoU mutant was more sensitive to acid pH 4.0 than the wild type for the stationary phase bacteria. After 7 day exposure to acid, no viable bacteria were recovered from the PhoU mutant, whereas the wild type had about 10⁸ CFU/ml from an original culture (FIG. 3B). The defect in survival at acid pH4.0 for the PhoU mutant was restored by complementation with the functional phoU gene whereas the PhoU mutant transformed with the vector control remained as susceptible as the mutant itself (FIG. 3B).

The PhoU mutant was also more susceptible to energy inhibitors DCCD (5 mM) (an FIFO ATPase inhibitor) and CCCP (1 mM) (a proton carrier that dissipates proton motive force) than the wild type strain W3110. After 1 day of exposure, there was a about 1000-fold drop in CFU counts in the PhoU mutant over that of the wild type strain W3110 in MOPS minimal medium at pH 5.0 (FIG. 3C).

The PhoU mutant was also more sensitive to hydrogen peroxide than the wild type W3110, and complementation of the PhoU mutant with the functional phoU gene restored peroxide resistance (Table 4). Under anaerobic conditions, the PhoU mutant was more susceptible than the wild type strain with about 100-fold less viable bacteria after 3 day incubation.

Susceptibility of the PhoU mutant and the wild type strain W3110 to weak acids was tested by incorporating SA (80 μg/ml) and pyrazinoic acid (230 μg/ml) gimp into LB agar at acid pH 5.0 in an MIC type of experiment when the growth inhibition was assessed by visible growth after incubating the LB plates at 37° C. overnight. The PhoU mutant was more sensitive to weak acids salicylic acid (80 μg/ml) and pyrazinoic acid (230 μg/ml) at pH 5.0 as shown by lack of growth at ⅓ MIC compared with the wild type strain W3110, which was resistant under such conditions (data not shown).

### Example 9

### Mutation of PhoU in E. coli Results in Increased Susceptibility to the Tuberculosis Drug PZA

Since weak acid susceptibility in M. tuberculosis is correlated with susceptibility to the frontline tuberculosis drug pyrazinamide (PZA) (a weak acid pyrazinoic acid amide) (Y. Zhang, H. Zhang, Z. Sun, J. Antimicrob. Chemother. 52, 56 (2003)), a persister drug that depletes membrane energy, kills non-replicating persister tubercle bacilli and shortens the TB therapy (Y. Zhang, D. A. Mitchison, int. J. Tuberc. Lung Dis, 7, 6 (2003)). The activity of TB persister drug pyrazinamide (PZA) is pH dependent and its activity is best evaluated in drug exposure type of experiment (M. M. Wade, Y. Zhang, J Antimicrob. Chemother 58, 936 (2006)). The susceptibility of the PhoU mutant and the wild type strain W3110 to PZA. (0.5 mg/ml and 2 mg/ml) was evaluated in a drug exposure type of experiment in minimal medium MOPS (pH 5.0) over a period of 7-10 days at 37° C. without shaking. Aliquots of bacterial cultures exposed to PZA were taken at different time points and washed in saline before plating for viable bacteria (CFU) on LB plates.

Interestingly, the stationary phase PhoU mutant was more susceptible to PZA (500 μg/ml, at pH 5.0 in MOPS minimal medium) than the wild type strain W3110 (FIG. 4A). The PhoU mutant and wild type strain had similar beginning CFU (10⁹/ml) and there was little difference in CFU counts between the two strains in the first 3 day incubation with PZA (FIG. 4A). However, upon extended incubation, the PhoU mutant had no viable bacteria left after 1 week, whereas the wild type strain had 5×10⁴ CFU/ml (FIG. 4A).

To determine if there is any difference between log phase and stationary phase cultures of the PhoU mutant and the wild type strain W3110, a drug exposure experiment with PZA (2 mg/ml) in MOPS minimal medium (pH 5.0) was performed. The stationary phase PhoU mutant was much more susceptible to PZA and was completely sterilized at day 6 whereas the stationary phase wild type strain W3110 had 6.7×10⁶ CFU/ml remaining (FIG. 4B). The log phase PhoU mutant was less susceptible to PZA than the stationary phase PhoU mutant, but was more susceptible than the log phase wild type strain W3110, such that by day 10, log phase PhoU mutant was completely killed whereas the log phase wild type W3110 had about 10⁶ CFU/ml left (FIG. 4). These findings are surprising, considering that normal growing E. coli is highly resistant to PZA with MIC>2 mg/ml at pH 5.0 in this type of experiment.

### Example 10

### PhoU Expression is Regulated by Nutrient Availability

To determine how PhoU, which is involved in persister formation, is regulated in response to changes in nutrient availability and during different growth phases, Western blot analysis was performed to monitor the expression of PhoU protein under these conditions. It was found that PhoU was not expressed, or expressed at a very low level during nutrient sufficiency in rich medium LB medium (FIG. 5). However, PhoU was highly expressed in nutrient limiting condition in minimal medium (FIG. 5). In addition, as the culture grew to stationary phase in LB medium, there was a slight increase in PhoU expression compared with the log phase culture presumably due to nutrient limitation in stationary phase (FIG. 5). These findings are consistent with the previous observation that pstSCAB-phoU operon expression is influenced by nutrient availability and age of bacteria.

### Example 11

### Mutation of PhoU Result in Variation in the Expression of About 350 Genes

PhoU is a global negative regulator beyond its role in phosphate metabolism PhoU is known to be a negative regulator of the Pho regulon, which consists of about 40 genes involved in phosphate metabolism. Phosphate starvation or mutation in PhoU leads to activation of the two-component system sensor PhoR which in turn activates the transcription factor PhoB to turn on the Pho regulon genes. However, the exact function of PhoU is not well understood.

DNA microarray analysis and qRT-PCR. The Affymetrix E. coli Genome 2.0 array was used in DNA microarray analysis of the PhoU mutant with the wild-type strain W3110 as a control. The PhoU mutant and the wild-type strain were grown in MOPS minimal medium overnight, and the RNA was isolated using a MasterPure RNA purification kit and reverse transcribed for making probes for array hybridization. The array was performed according to the manufacturer's instructions at the Johns Hopkins Malaria Research Institute Gene Array Core Facility. Triplicate samples of the PhoU mutant and the wild-type strain W3110 were used for each individual array (six arrays total), and the array data were analyzed using SAM (significance analysis of microarrays) software. For quantitative real-time PCR (qRT-PCR), the SuperScript III Platinum SYBR green one-step qRT-PCR kit was used. For qRT-PCR, the phoU primers were 5′-TAT TGG CGA CGT GGC GGA C-3′ (SEQ ID NO: 10) and 5′-ATG AAT GAC GCG ACA AGA CG-3′ (SEQ ID NO: 11); the phoE primers were 5′-TCA ACT GAC TGG TTA TGG TCG-3′ (SEQ ID NO: 12) and 5′-TGT TGA AAT ACT GGT TTG CGC-3′ (SEQ ID NO: 13); and the fliA primers were 5′-ACT TGA CGA TCT GCT ACA GG-3′ (SEQ ID NO: 14) and 5′-TAG CGG TTT ACA ACG AGC TG-3′ (SEQ ID NO: 15).

A total of about 350 genes were up-regulated by at least two fold, and many of these genes with known functions are listed in Table 3. As expected, genes involved in phosphate metabolism (phoE, phoA, phoB, phoR, pstS, pstC, pstA, pstB, phnC, phnD, psiF, ugpB etc) were induced in the PhoU mutant due to inactivation of PhoU as a negative regulator. Surprisingly, genes involved in energy production (sdhBD, nuo operon genes, atpB, acnB, nidh, ugpC,E, cyoA,B, etc), some membrane transporters of various nutrients (pro V, X artJ, fiu, pro′JkptP, livJ, hisJ, copA, aroP, yhgL, yaeC, yebM, yicE, gltl, livG, oppA, meth, 1,7; trxB, sect, fhuE, cusF, B, X), transcription factors (ArcA, PdhR, FlhD, Befl, OsmE, Feel, SoxS, SspA (stringent starvation protein A, a global regulator associates with RNA polymerase), and regulatory RNA (small antisense RNA SgrS), and in particular genes involved in flagella synthesis (over 40 flagella genes) and chemotaxis, were upregulated at a much higher level in the PhoU mutant compared with the wild type (Table 3). An increase in expression of at least 5-fold was observed for the Pho regulon gene phoA, the flagellar gene fliA, the small antisense RNA SgrS, and the metabolic enzyme purK. An increase in expression of at least 10-fold was observed for the Pho regulon gene phoE.

These findings suggest PhoU is a global negative regulator beyond its role as a negative regulator of Pho regulon in phosphate metabolism, whose inactivation leads to a metabolically hyperactive status of the cell. The very striking induction of numerous flagella and chemotaxis genes along with increased expression of energy production enzymes in the PhoU mutant suggest that loss of the PhoU function makes the cells more active as if the cells were trying to “escape” or seek for nutrients. The high metabolic status of the PhoU mutant may be advantageous for the cells in a short term but in the long term may be at a disadvantage due to high consumption of energy especially in the nutrient limiting or stress conditions such as starvation.

The highly metabolically active status of the cells provides an explanation for why the PhoU mutant is more susceptible to various antibiotics and stresses. This is because loss of the PhoU as a negative regulator causes the PhoU mutant to lose the ability to suppress the metabolic processes necessary for persister formation; therefore, no persisters could be produced making the cells without PhoU are more sensitive to stresses and antibiotics. The finding that increased expression of energy production and flagella and chemotaxis genes in PhoU mutant is also consistent with the previous observation that E. coli persisters had decreased expression of energy production genes and flagella genes.

### Example 12

### Persister Formation is Independent of PhoR-PhoB Two-Component System

To confirm that the disruption of phoU gene was responsible for the decrease in persister formation, a deletion mutant, ΔphoU was generated using standard methods. Wild type W3110, the PhoU transposon mutant identified in the library screen, and the PhoU deletion were tested for susceptibility to ampicillin (100 μg/ml) and norfloxacin (3 μg/ml) in Example 5. In log phase cultures, killing by ampicillin was most rapid in the JHU-313 culture transposon mutant, somewhat less rapid in the phoU deletion mutant, and slowest in the W3110 bacteria (Table 5). However, all cells were killed by three days.

In the stationary phase, both the phoU transposon disruption and deletion mutant showed complete killing by day 3, whereas the wild type culture remained viable throughout the time of the experiment (10 days). Killing by norfloxacin was substantially more rapid in the log phase of both phoU mutants as compared to the wild type bacteria (1 day vs. 5 days). Killing of the stationary cultures was complete by 5 days in both phoU mutant bacterial cultures, whereas viable bacteria persisted in the wild type culture, although substantially reduced from the CFU of the original culture, at the end of the experiment (10 days).

As expected, PhoU deletion mutant had the same persister deficiency phenotype as the PhoU transposon insertion mutant JHU-313 for both ampicillin (100 μg/ml) and norfloxacin (3 μg/ml) (Table 5). However, the PhoU deletion mutant grew more poorly than the PhoU transposon mutant and was not stable as reported previously (P. M. Steed, B. L. Wanner, J. Bacteriol. 175, 6797 (1993)). However, inactivating PhoB or PhoR did not affect persister formation in E. coli (data not shown). This suggests that persister formation is not dependent on the PhoR-PhoB two component system and supports the array data herein that PhoU has the additional function of persister formation independent of its role in regulation of phosphate metabolism. Inactivation of the known persister gene hipA had no apparent effect on persister formation, which is consistent with multiple independent pathways for persister formation.

### Example 13

### Mutation of PhoU Homolog PhoY1 and PhoY2 Alters Susceptibility of M. tuberculosis M37Rv to TB Drugs in MIC/MBC Tests and in Drug Exposure Assays

Minimum inhibitory concentrations of the tuberculosis agents isoniazid (INH) and rifampin (RIF) on wild type and mutant PhoY1 and PhoY2 M. tuberculosis strain M37Rv were determined by using serial twofold dilution of the compound in 7H9 medium and on 7H11 agar. MIC of pyrazinamide (PZA) was determined by using serial twofold dilution of the compound in 7H9 medium pH5.6 (Table 6A) and on 7H11 agar pH5.6 (Table 6B). The initial cell density was 10⁵ cell/ml of log phase cultures, and the samples were incubated for 10 days at 37° C. The MIC was recorded as the minimum drug concentration that prevented visible growth, and the MBC was recorded as the drug concentration that reduced colony forming units (CFU) by 100-fold over the seeded inoculum.

Mutation of PhoY1 resulted in a small increase susceptibility to INH and RF as compared to wild type M37Rv in the MIC analysis in liquid culture. No corresponding increase in susceptibility was observed in the MIC and MBC assays performed on plates. Mutation of PhoY2 resulted in an increase in susceptibility to all pharmaceutical agents under both growth conditions. This demonstrates that PhoU is involved in persister formation in multiple bacterial types.

### Example 14

### Mutation PhoY2 Alters Susceptibility of M. tuberculosis M37Rv to TB Therapeutic Agents Reduces Persister Formation

The susceptibility of stationary phase cultures of the PhoY1 and PhoY2 mutants and the parent strain H37Rv to pyrazinamide (PZA), rifampin (RIF), was evaluated in a drug exposure assay. The drug exposure was carried out over a period of 3 to 10 days at 37° C. without shaking. Aliquots of bacterial cultures exposed to drugs were taken at different time points and washed in PBS buffer before plating for viable counts (CFU) on 7H11 agar plates. It was found that PhoY1 and PhoY2 mutants were initially killed as much as the wild type strain H37Rv during the first 3 days but the PhoY2 mutant showed higher susceptibility to PZA and RIF at 9 day exposure. PhoY1 mutant did not have significant difference compared to the parent strain H37Rv in the drug exposure assay (Table 7). The above studies demonstrate that PhoY2 results in an increase in multiple drug sensitivities in M. tuberculosis as in E. coli.

### Example 15

### PhoU and PhoY2 have Kinase Activity

In order to determine if PhoU has kinase-like activity, autophosphorylation of purified E. coli PhoU, PhoB, PhoR were tested. The PhoU proteins were expressed as recombinant tagged protein and purified using methods known to those in the art. The purified PhoU, PhoB and PhoR were incubated in the presence of [γ-³²P]ATP in the assay. Proteins were separated on 14% SDS-PAGE. Sizes and the amount of loaded proteins were determined relative to molecular size markers that stained on the sodium dodecyl sulfate-polyacrylamide gel (FIG. 6A), which was subsequently exposed to film for autoradiography (FIG. 6B). A single strong radioactively labeled band of approximately 27-kDa of PhoU was observed (FIG. 6B, indicated by arrow), whereas PhoR showed a weaker band. In contrast PhoB did not show phosphorylation. The co-migration of the 50-kDa radiolabeled band correlated with purified His-PhoR, and comigration of the 27-kDa radiolabeled band correlated with purified His-PhoU. These data suggest that PhoU, like PhoR, known to be a histidine kinase, has an even stronger kinase activity than PhoR.

### Example 16

### PhoU has Phosphatase Activity

PhoU was suspected of having phosphatase activity. In order to test PhoU for phosphatase activity, purified PhoU protein was analyzed using the EnzChek phosphatase assay kit (Invitrogen, Molecular Probes). Phosphatase activity was continuously monitored at acidic pH 5.5 and 6,8-difluoro-4-methylumbelliferyl phosphate (DiFMUP) was used as the substrate according to manufacturer's instruction.

Result presented in FIG. 7 suggests that PhoU has an acid phosphatase activity at acidic pH 5.5. However, PhoU had no obvious phosphatase activity at neutral and alkaline pH conditions (data not shown).

### Example 17

### PhoU Demonstrates Phosphatase Substrate Specificity

Different substrates for the PhoU phosphatase activity were tested. In vivo signal receiver domain could be autophosphorylated using acetyl phosphate (acetyl-P), carbamoyl phosphate (carbamoyl-P) as phosphodonors. For PhoU, the substrates acetyl-P, carbamoyl-P, imido-di phosphate (imido-di-P), r-nitrophenyl phosphate (rNPP), β-Nicotinamide adenine dinucleotide phosphate (NADP), Glucose-1,6-bisphosphate (Glucose-1,6-bisP) were tested at pH5, pH7, and pH8 condition, as potential PhoU phosphatase activity. Good substrates are in the following order: acetyl-P>rNPP>carbamoyl-P>Glucose-1,6-bisP>NADP. And imido-di-P was poorest substrate (data not shown). In general, the phosphatase activity of PhoU was higher using the above substrates at acidic pH but lower activity at neutral pH or alkaline pH.

### Example 18

### Phosphatase Activity of PhoU is Affected by the Presence and Identity of Specific Cations

Since most phosphatases have a metal requirement for activity, using either Mg²⁺ or Zn²⁺, the effects of various metal ions on the PhoU phosphatase enzyme activity were examined at acidic pH 5.5 and using DiFMUP as a substrate. They include Ca²⁺, Cu²⁺, Mn²⁺, Mg²⁺, Zn²⁺, Fe²⁺, and Fe³⁺. PhoU was activated over 10 fold by 500 μM Fe³⁺ (FIG. 8). All other ions including Fe2+ did not stimulate the phosphatase activity of PhoU. This result is consistent with the finding that the crystal structure of PhoU protein from Thermotoga maritima, which is metalloprotein, contains multinuclear iron clusters.

### Example 19

### Effect of PhoU Mutations on Phosphatase Enzyme Activity and Persister Formation

In order to study PhoU protein function, PhoU mutants, including deletion and point mutations in the PhoU protein coding sequence were constructed in an expression vector. PhoU deletion constructs include: pPhoU80 and pPhoU118, which contain 80 and 118 amino acids of PhoU protein, respectively. The point mutations were introduced by site-directed mutagenesis that altered A51H and G219H of the PhoU. pPhoUG219H was designed to change the amino acid at the transposon insertion site from our transposon PhoU mutant JHU313 (Li and Zhang, Antimicrob. Agents Chemo. 51:2092-2099 (2007), incorporated herein by reference), which we found to be involved in a molecular switching in bacterial persister formation.

Plasmids were transformed into E. coli PhoU deletion mutant W3110ΔphoU. The W3110 wild type strains and PhoU mutant strains were grown in MOPS minimal medium overnight. The cells were washed by MOPS medium without phosphate, and then resuspended in MOPS alone, with low phosphate (0.01 mM) or sufficient phosphate (2 mM), in the presence or absence of iron (Fe³⁺ 0.25 mM). The exposure to ampicillin (100 ug/ml) was done in the presence or absence of phosphate and iron, since they have been shown to affect PhoU phosphatase activity as described above.

The results (Table 8) showed that cells grown at higher phosphate concentration (2 mM) condition gave higher sensitivity to ampicillin compared with cells grown in low phosphate concentration (0.01 mM). Cells with overexpressed PhoUG219H showed highest sensitivity to ampicillin exposure at higher phosphate condition compared to other strains, while cells with overexpressed PhoU118 gave the least sensitivity to ampicillin under the same phosphate concentration. When cells were grown in the presence of Fe³⁺, the sensitivity to ampicillin of the PhoUG219H disappeared, but the sensitivity of PhoU80 to ampicillin increased.

### Example 20

### Kinetic Analysis of PhoU Phosphatase Activity Wild Type and Mutant E. coli PhoU

Kinetic assays were performed to further characterize PhoU phosphatase activity. For comparison of enzyme activity, a fixed amount of protein (0.2 nM) for each mutant protein was added in the reactions with four different concentrations (100, 50, 25, 12.5 uM) of substrate 6,8-difluoro-4-methylumbelliferyl phosphate (DiFMUP). The reaction rate was measured by recording the absorbance at 360/465 nm at 1-minute intervals for 10 minutes. The Lineweaver-Burk plot was used to calculate the Kₘ and Vₘₐₓ (Table 9).

PhoU80, which contains only the N-terminal 80 amino acids of PhoU protein, had no phosphatase activity. PhoU118, which contains one PhoU domain, half of PhoU protein, gave some phosphatase activity. This means that the single PhoU domain has at least some phosphatase activity (see, FIG. 1G). Point mutation protein PhoUG219H had lower activity, but Vₘₐₓ was increased.

### Example 21

### PhoU Phosphatase Activity is Increased by Fe³⁺ but Inhibited by Phosphate

Phosphatase assays were performed using purified recombinant wild type and mutant PhoU proteins. In the presence of Fe³⁺, PhoU and PhoU118 was activated, and showed the highest phosphatase activity (FIG. 9A), compared to PhoU and PhoUG219H. Phosphate could inhibit the phosphatase activity as seen from the FIG. 4B, and Fe³⁺ could not reverse this inhibition by phosphate (data not shown). These changes in kinetics parameters, in conjunction with changes in sensitivity to phosphate and cation concentrations means that modulation of such factors in the bacteria can alter persister formation.

### Example 23

### M. tuberculosis PhoU Homologs PhoY2 and PhoY1 have Phosphatase Activity which Can be Stimulated by Fe³⁺ and Inhibited by Phosphate

The phosphatase activity of both PhoY1 and PhoY2 of M. tuberculosis were tested in the presence of Fe²⁺, Fe³⁺, and inorganic phosphate using the EnzChek kit as above.

M. tuberculosis PhoY1 and PhoY2 have high phosphatase activities at acidic pH 5.5. The presence of Fe³⁺ greatly stimulated the phosphatase activity of PhoY1 and PhoY2, whereas Fe²⁺ had much less ability to increase phosphatase activity. In contrast, phosphate inhibited PhoY1 and PhoY2 activity (FIG. 10A, B), a finding that is similar to the effect of Fe³⁺ and phosphate on E. coli PhoU. These data suggest that agents that inhibit phosphatase activity of E. coli PhoU protein will inhibit phosphatase activity of PhoY1 and PhoY2.

### Example 24

### PhoU Phosphatase Inhibitors were Identified from the FDA Approved Compound Library for Inhibitors

The FDA approved compound library consisting of about 3000 clinical compounds (50 μM final concentration) was screened for inhibitory activity for M. tuberculosis PhoU homolog PhoY2 for phosphatase activity using the EnzChek phosphatase assay kit in a 96 well plate format. The assay was run for 30 min, 60 min and overnight when the plates were read in a fluorescence plate reader (Perkin Elmer). Aurintricarboxylic acid, a known phosphatase inhibitor, was included as a positive control. Four hits, piperazine, pyrantel pamoate, meclocycline and doxycycline were found to inhibit PhoY2 enzyme activity (FIG. 11).

### Example 25

### PhoY2 Inhibitors Minocycline and Pyrantel Tartrate on Increasing the Activity of TB Drugs Isoniazid, Rifampin and Pyrazinamide for M. tuberculosis H37Rv

PhoY2 phosphatase activity inhibitors identified in the screen were tested for their ability to decrease persister formation in M. tuberculosis. The effect of PhoY2 inhibitors minocycline and pyrantel tartrate to increase killing in conjunction with the TB drugs isoniazid, rifampin and pyrazinamide for M. tuberculosis H37Rv. Old stationary phase cells of H37Rv (starting CFU: 1.3×10⁸) were exposed to drug 1 (Minocycline), drug 2 (Pyrantel tartrate) alone and in combination with INH, RIF or PZA at 37° C. After one and two week exposure, CFU (colony forming unit) was measured. It can be seen that the PhoY2 inhibitors could enhance the activity of INH, RIF or PZA against M. tuberculosis H37Rv (Table 10). In particular, PhoY2 inhibitor minocycline was more active than pyrantel in enhancing INH, RIF, and PZA activity. It can be envisioned that PhoY2 inhibitors by inhibiting persister bacteria could enhance the activity of current TB drugs INH, RIF and PZA and may be used in combination with current TB drugs for improved treatment of TB.

### Example 26

### Identification of Agents that Reduce Expression or Activity of PhoU in a PhoU Containing Bacteria

Disruption or deletion of PhoU in a bacteria results in a decrease in persister formation. A screen for agents that disrupt transcription and/or translation of PhoU can be accomplished using library screening methods. For example, stationary phase PhoU containing bacteria cultured in an antibiotic to which the bacteria are resistant (e.g., kanamycin or ampicillin resistance can easily be transferred to bacteria by transformation), are aliquotted into triplicate multiwell plates. Bacteria in the duplicate plates are contacted with agents from a library or a vehicle control, and optionally incubated for a predetermined time. The agent, for example, can be an agent from the library of FDA approved compounds used above. The bacteria in one of the two of the duplicate plates is further contacted with an antibiotic. The plates are incubated in parallel and differences in cell viability between corresponding wells in the two plates is determined, and the cell viability between the control antibiotic treated bacteria with the agent treated bacteria is determined. Agents that result in increased killing upon exposure to antibiotic relative to the vehicle control antibiotic treated bacteria, that do not increase killing in the absence of exposure to antibiotic can be involved in persister formation. Lack of growth can be determined by optical density, for example, using a plate reader. Such agents can be analyzed and characterized using assays and methods such as those described herein to determine if they act through PhoU. Agents that kill both cultures equally likely have antibiotic activity.

### Example 27

### Identification of Agents that Inhibit Expression of phoU

PhoU activity can be inhibited by inhibiting transcription or translation of the phoU gene or PhoU protein, respectively. Methods for screening for inhibitors of transcription are well known and typically involve the use of a reporter construct operably linked to the transcriptional regulatory region of the gene. In bacteria, transcriptional regulation of a corresponding gene can be controlled by one or more transcriptional control regions including the regulon, the operon, and the promoter region. Many regulon, operon, and promoter sequences have been mapped in bacteria. Programs such as Virtual Footprint and PRODORIC (Munch et al, Bioinformatics. 21:4187-4189, incorporated herein by reference). The programs can also be used to identify putative transcriptional binding sites which may assist in the identification of agents that can inhibit transcription from the transcriptional regulation region.

Using methods well known to those in the art, the transcriptional control region can be functionally linked to a reporter gene to form a reporter construct, typically on a plasmid including an antibiotic resistance gene. Alternatively, the reporter construct can be inserted into the bacterial genome using methods known to those skilled in the art. It is preferred that the transcriptional control sequence in the reporter construct is distinct from transcriptional control regions in the bacteria in which the reporter construct is inserted. The reporter construct is tested in the bacteria to insure that transcription from the reporter construct occurs in under normal growth conditions.

The bacteria including the reporter construct are grown, preferably to less than stationary phase, preferably to a point in log phase, preferably low log phase in the presence of the antibiotic resistance gene included in the reporter construct. Cells are aliquotted into multiwell plates and exposed to an agent, such as an agent from a library. Cells are grown for a predetermined amount of time and expression of the gene from the reporter construct is detected. Reporter genes for use in constructs typically include luciferase, which results in the production of a fluorescent product upon exposure to an appropriate substrate, and β-galactosidase, which results in the production of any of a number of colored products depending on the substrate, typically a blue product. Kits and methods for the detection of such products is well known to those of skill in the art. Agents that decrease expression from the reporter construct, preferably on a per mass quantity of bacterial extract, can potentially inhibit the transcription of phoU in a cell. Agents identified using the screen can be further characterized using assays described herein, such as killing assays.

### Example 28

### Identification of Agents that Cause an Increase in Intracellular Bacterial Phosphate or Sensitivity to Phosphate

PhoU phosphatase activity can be inhibited by increased intracellular phosphate. Methods for detection of agents that result in an increase in intracellular phosphate or result in an increase in phosphate sensitivity can be identified using library screening methods. For example, stationary phase PhoU containing bacteria cultured in an antibiotic to which the bacteria are resistant (e.g., kanamycin or ampicillin resistance can easily be transferred to bacteria by transformation), are aliquotted into duplicate multiwell plates. One of the duplicate plates of bacteria (e.g., A and B) are grown in the presence of inorganic phosphate at a concentration lower than that which inhibits PhoU phosphatase activity alone (e.g., at 1 mM, 0.5 mM, 0.2 mM, 0.1 mM, tolerance inhibition of PhoU activity by inorganic phosphate may vary between bacterial types).

Bacteria in duplicate plates are contacted with agents from a library or a vehicle control and optionally incubated for a predetermined time. The agent, for example, can be an agent from the library of FDA approved compounds used above. The bacteria in both of the duplicate plates is further contacted with an antibiotic. The plates are incubated in parallel and differences in cell viability between corresponding wells in the two plates is determined, and the cell viability between the control antibiotic treated bacteria with the agent treated bacteria is determined. Agents that result in increased killing upon exposure to antibiotic relative to the vehicle control antibiotic treated bacteria, that do not increase killing in the absence of exposure to antibiotic can be involved in persister formation. Lack of growth can be determined by optical density, for example, using a plate reader. Such agents can be analyzed and characterized using assays and methods such as those described herein to determine if they act through PhoU. Agents that kill both cultures equally likely have antibiotic activity.

TABLE 1

| | | | | JHU-313 |
|---|---|---|---|---|
| | | | JHU-313 | containing |
| Antibiotics | W3110 | JHU-313 | containing pPhoU | pVector |
| Ampicillin | 3.1/12.5 | 1.5/6.25 | 3.1/12.5 | 3.1/6.25 |
| Gentamicin | 2.5/5 | 1.25/2.5 | 2.5/5 | 1.25/2.5 |
| Trimethoprim | 2/8 | 0.25/1 | 2/4 | 0.5/1 |
| Norfloxacin | 0.5/1 | 0.125/0.5 | 0.5/1 | 0.125/0.5 |

TABLE 2

| | | | PhoU mutant (JHU-313) |
|---|---|---|---|
| Time of antibiotic exposure | | W3110 (CFU/mI) | (CFU/ml) |
| Starting CFU | | 5 × 10⁷ | 1.4 × 10⁷ |
| 1.5 h | Ampicillin-treated | 7 × 10⁴ | 4 × 10⁴ |
| 20 h | Ampicillin | 7 × 10⁵ | 0 |
| | Gentamicin | 5 × 10⁵ | 2 × 10³ |
| | Trimethoprim | 6 × 10⁵ | 6 × 10⁵ |
| | Norfloxacin | 7 × 10⁵ | 0 |

TABLE 3

| Genes | Description |
|---|---|
| phoE, A, U, B, R, H | Pho regulon genes |
| pstS, B, A, C | |
| phnC, D | |
| psiF, ugpB | |
| fliA, C, D, E, F, G, H, I, J, K, L, | Flagellar genes |
| M, N, 0, P, Q, R, S, T, Z | |
| flgA, B, C, E, F, G, H, D, I, | |
| K, J, L, M, N | |
| motA, B | |
| flhB, C, A, D, E | |
| cheA, B, W, Y, R | Chemotaxis genes |
| aer, trg, tar, tsr | |
| arcA, cheA, cheZ, yoeB | Two component systems and |
| | toxin-antitoxin (TA) modules |
| sgrS, betl, spoT, malT, stpA, glnK, | Regulators, repressor and small |
| yefM, ycfQ, crl, rtT, isrB, | RNA |
| rpsU, iscR, | |
| iscU, iclR, trpR, sspA | |
| proV X, artJ, fiu, pro W, kptP, IivJ, | Transporter systems |
| hisJ, copA, aroP, yhgL, | |
| yaeC, yebM, | |
| yicE, gltl, livG, oppA, metN, l, | |
| T, trxB, secG, fhuE, | |
| cusF, B, X | |
| purK, E, M, D, N, C, F, L, H, B | Metabolic enzymes |
| carA, B | |
| nuoA, B, C, E, F, G, M | |
| argG, A, D, C | |
| cyoA, B | |
| sdhA, D, C, B | |
| pyrB, I, D, C, L | |
| sucA, B, C | |
| aceB, E | |
| nrdF, I, H, E | |
| sodA, B | |
| yeaA, F | |
| aroL. ilvC, acnB, aceA, folE, | |
| en,. yojH, fadA | |

TABLE 4

| Antibiotic | | | | JHU-313 |
|---|---|---|---|---|
| concentrations | | | JHU-313 | containing |
| (μg/ml) | W3110 | JHU-313 | containing pPhoU | pVector |
| Ampicillin | | | | |
| 100 | 35 | 40 | 40 | 45 |
| 25 | 28 | 35 | 36 | 40 |
| 6 | 25 | 30 | 32 | 34 |
| 1.5 | 21 | 25 | 25 | 27 |
| Gentamicin | | | | |
| 10 | 25 | 27 | 28 | 31 |
| 2 | 22 | 26 | 24 | 30 |
| 0.5 | 20 | 22 | 22 | 29 |
| 0.1 | 14 | 20 | 17 | 22 |
| Tetracycline | | | | |
| 50 | 23 | 32 | 32 | 34 |
| 25 | 23 | 32 | 32 | 34 |
| 12.5 | 22 | 26 | 30 | 34 |
| Trimethoprim | | | | |
| 2 | 30 | 42 | 36 | 44 |
| 1 | 26 | 38 | 33 | 40 |
| 0.5 | 24 | 31 | 32 | 36 |
| Norfloxacin | | | | |
| 4 | 32 | 37 | 36 | 44 |
| 2 | 30 | 34 | 35 | 40 |
| 0.5 | 27 | 30 | 31 | 35 |
| 0.1 | 24 | 28 | 27 | 29 |
| Hydrogen | 30 | 37 | 39 | 46 |
| peroxide (30%) | | | | |

TABLE 5

| | | PhoU mutant transposon | | | |
|---|---|---|---|---|---|
| W3110 | | mutant) (JHU-313 | | ΔphoU | |
| Log | Stationary | Log | Stationary | Log | Stationary |
| phase | phase | phase | phase | phase | phase |

Ap100

| | | | | | | |
|---|---|---|---|---|---|---|
| Start CFU | 4 × 10⁸ | 7 × 10⁹ | 3 × 10⁸ | 4 × 10⁹ | 4 × 10⁸ | 7 × 10⁹ |
| 5 hr | 3 × 10³ | 5 × 10⁹ | 20 | 2 × 10⁸ | 6 × 10² | 6 × 10⁸ |
| 1 day | 10 | 3 × 10⁹ | 0-1 | 1 × 10⁷ | 0-1 | 1 × 10⁸ |
| 3 days | 0 | 1 × 10⁸ | 0 | 0 | 0 | 0 |
| 5 days | 0 | 3 × 10⁷ | 0 | 0 | 0 | 0 |
| 1 week | 0 | 5 × 10⁶ | 0 | 0 | 0 | 0 |
| 10 days | 0 | 2 × 10⁵ | 0 | 0 | 0 | 0 |

Norf3

| | | | | | | |
|---|---|---|---|---|---|---|
| 5 hr | 1 × 10⁸ | 5 × 10⁹ | 2 × 10⁷ | 3 × 10⁸ | 5 × 10⁷ | 6 × 10⁸ |
| 1 day | 4 × 10⁶ | 4 × 10⁹ | 4 × 10⁵ | 4.5 × 10⁷ | 1 × 10⁶ | 2 × 10⁸ |
| 3 days | 5 × 10⁴ | 1 × 10⁸ | 0 | 2 × 10⁵ | 0 | 3 × 10⁵ |
| 5 days | 7 × 10² | 4 × 10⁶ | 0 | 0 | 0 | 0 |
| 1 week | 0 | 1.5 × 10⁴ | 0 | 0 | 0 | 0 |
| 10 days | 0 | 2 × 10² | 0 | 0 | 0 | 0 |

TABLE 6A

| | H37Rv MIC | PhoY1 MIC | PhoY2 MIC |
|---|---|---|---|
| drug | (μg/ml) | (μg/ml) | (μg/ml) |
| PZA pH5.6 | 200 | 200 | 100 |
| INH | 0.2 | 0.1 | 0.1 |
| RIF | 0.2 | 0.1 | 0.1 |

TABLE 6B

| | strain | | | | | |
|---|---|---|---|---|---|---|
| Drug | H37Rv | | PhoY1 | | PhoY2 | |
| (μg/ml) | MIC | MBC | MIC | MBC | MIC | MBC |
| PZA | 200 | 400 | 200 | 400 | 100 | 400 |
| pH 5.9 | | | | | | |
| RIF | 0.1 | 0.2 | 0.05 | 0.2 | 0.025 | 0.05 |

TABLE 7

| | | Concen- | | | | |
|---|---|---|---|---|---|---|
| | | tration | | | CFU/ml | CFU/ml |
| condition | drug | μg/ml | strain | start | 3 day | 9 day |
| pH 5.6 | PZA | 200 | H37Rv | 3.1 × 10⁶ | 3.1 × 10⁵ | 5.3 × 10³ |
| 7H9 | | | PhoY1 | 3.2 × 10⁶ | 4.3 × 10⁵ | 3.3 × 10³ |
| No ADC | | | PhoY2 | 2.4 × 10⁶ | 4.83 × 10⁵ | <10² |
| | — | | Rv | 3.1 × 10⁶ | 6.9 × 10⁵ | 1.33 × 10⁵ |
| | — | | PhoY1 | 3.2 × 10⁶ | 2.07 × 10⁶ | 3.97 × 10⁵ |
| | — | | PhoY2 | 2.4 × 10⁶ | 4.77 × 10⁵ | 2.77 × 10⁵ |
| neutral | RIF | 8 | H37Rv | 3.1 × 10⁸ | 3.37 × 10⁵ | 1.67 × 10⁴ |
| pH 7H9 | | | PhoY1 | 3.2 × 10⁸ | 4.97 × 10⁵ | 3.13 × 10⁴ |
| | | | PhoY2 | 2.4 × 10⁸ | 2.27 × 10⁵ | <10² |
| | — | | H37Rv | 3.1 × 10⁸ | 1.33 × 10⁹ | 3.5 × 10⁸ |
| | — | | PhoY1 | 3.2 × 10⁸ | 2.0 × 10⁹ | 3.57 × 10⁸ |
| | — | | PhoY2 | 2.4 × 10⁸ | 1.37 × 10⁹ | 3.0 × 10⁸ |

TABLE 8

| Strains | | | | | | Pi 0.01 mM | Pi 2 mM |
|---|---|---|---|---|---|---|---|
| (induced by | Start | | Pi | Pi | Fe3 + | Fe3 + | Fe3 + |
| 1 mM IPTG ) | CFU | N/A | 0.01 mM | 2 mM | 250 uM | 250 uM | 250 uM |
| W3110 | 5 × 10⁸ | 5 × 10⁸ | 3 × 10⁸ | 9 × 10⁶ | 5 × 10⁸ | 5 × 10⁸ | 5 × 10⁸ |
| W3110Δ | 3 × 10⁸ | 1 × 10⁸ | 1 × 10⁷ | 1 × 10⁵ | 1 × 10⁸ | 1 × 10⁸ | 1 × 10⁷ |
| phoU | | | | | | | |
| W3110Δ | | | | | | | |
| phoU | | | | | | | |
| containing | | | | | | | |
| pET28a | 5 × 10⁸ | 3 × 10⁸ | 1 × 10⁸ | 1 × 10⁵ | 1 × 10⁸ | 1 × 10⁸ | 1 × 10⁸ |
| pPhoU80 | 5 × 10⁸ | 5 × 10⁶ | 2 × 10⁶ | 1 × 10⁵ | 1 × 10⁷ | 1 × 10⁵ | 0 |
| pPhoU118 | 5 × 10⁸ | 5.5 × 10⁸ | 5 × 10⁸ | 5 × 10⁸ | 5 × 10⁸ | 5 × 10⁸ | 5 × 10⁸ |
| pPhoUA51H | 5 × 10⁸ | 4 × 10⁸ | 3 × 10⁸ | 3 × 10⁵ | 1 × 10⁸ | 1 × 10⁸ | 1 × 10⁸ |
| pPhoUG219H | 5 × 10⁸ | 3 × 10⁸ | 2 × 10³ | 0 | 1 × 10⁸ | 1 × 10⁸ | 1 × 10⁸ |
| pPhoU | 5 × 10⁸ | 5 × 10⁸ | 3 × 10⁸ | 1 × 10⁷ | 3 × 10⁸ | 3 × 10⁸ | 3 × 10⁸ |

TABLE 9

| | | | |
|---|---|---|---|
| Proteins | Km (M) | Vmax (min⁻¹) | Vmax/Km (M⁻¹min⁻¹) |

| | | | |
|---|---|---|---|
| PhoU | 4 × 10⁻⁵ | 23.81 | 6 × 10⁵ |
| PhoU80 | 0 | 0 | 0 |
| PhoU118 | 1.7 × 10⁻⁴ | 23.81 | 1.4 × 10⁵ |
| PhoUG219H | 1.7 × 10⁻⁴ | 40 | 2.4 × 10⁵ |

Those skilled in the art will recognize, or be able to ascertain using no more than routine experimentation, many equivalents to the specific embodiments of the invention described herein. Such equivalents are intended to be encompassed by the following claims.

All references, patents, patent publications, and sequence reference numbers cited herein are incorporated herein by reference

1. A method to decrease persister formation and/or increase killing of a bacterial cell comprising contacting a PhoU containing bacteria with an agent that inhibits activity of a PhoU protein.

2. The method of claim 1, wherein the agent is an inhibitor of a PhoU phosphatase activity in a PhoU containing bacteria.

3. The method of claim 1, wherein the inhibitor of PhoU phosphatase activity is one or more of piperazine, pyrantel pamoate, tetracycline, meclocycline, doxycycline, aurintricarboxylic acid, or a PhoU specific antibody.

4. The method of claim 1, wherein the agent decreases PhoU phosphatase activity by decreasing expression of a PhoU phosphatase in a bacterial cell.

5. The method of claim 4, wherein the agent that decreases PhoU expression is an antisense oligonucleotide.

6. The method of claim 1 wherein the activity of a PhoU protein is inhibited by an agent that promotes an increase in intracellular bacterial inorganic phosphate or ATP.

7. The method of claim 1 wherein the activity of a PhoU protein is inhibited by an agent that promotes an increase in bacterial metabolic activity.

8. A method to decrease persister formation and/or increase killing of a bacterial cell comprising administration of an agent that increases metabolic activity and/or increases phosphate concentration in a bacteria.

9. The method of claim 8, wherein increased phosphate concentration in bacterial cells decreases PhoU phosphatase activity.

10. A method for identification of an agent that decrease persister formation and/or increase killing of a bacterial cell comprising:
contacting a PhoU phosphatase with an agent and
detecting a decrease in phosphatase activity of a PhoU phosphatase as compared to a control not contacted with an agent.

11. The method of claim 10, wherein the PhoU phosphatase is in a cell free system.

12-18. (canceled)

19. A method for amelioration or treatment of an infection with a bacteria to decrease persister formation and/or increase killing of a bacterial cell by administration of an agent that decreases phosphatase activity of a PhoU phosphatase.

20. The method of claim 19, wherein the agent comprises an inhibitor of a PhoU phosphatase activity in a PhoU containing bacteria.

21. The method of claim 19, wherein the inhibitor of PhoU phosphatase activity comprises one or more of piperazine, pyrantel pamoate, meclocycline, doxycycline, or aurintriccarboxylic acid.

22. The method of claim 19, wherein the agent decreases PhoU phosphatase activity by decreasing expression of a PhoU phosphatase in a bacterial cell.

23. A method of use of a PhoU phosphatase activity inhibitor comprising use of the PhoU phosphatase activity inhibitor as an adjuvant in combination with an antibacterial agent for the treatment of a bacterial infection.

24. (canceled)

25. The method of claim 23, wherein the infection is an E. coli infection.

26. The method of claim 23, wherein the bacterial infection is infection by Mycobacterium tuberculosis.

27-30. (canceled)

31. A composition comprising a pharmaceutically acceptable antibacterial agent in combination with a pharmaceutically acceptable PhoU phosphatase activity inhibitor.

32-33. (canceled)
